(19) United States
(12) Patent Application Publication
(10) Pub. No.: US 2001/0024664 A1
(43) Pub. Date: Sep. 27, 2001

(54) Selective COX-2 inhibition from edible plant extracts

(76) Inventors: Mark G. Obukowicz, St. Louis, MO (US); Susan L. Hummert, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/737,892

(22) Filed: Dec. 15, 2000

Related U.S. Application Data
(63) Continuation-in-part of application No. 09/272,363, filed on Mar. 19, 1999.

(51) Int. Cl. A61K 35/78

(52) U.S. Cl. 424/725; 424/757; 424/760; 424/752; 424/738; 424/754

(57) ABSTRACT

The present invention is directed toward a method for inhibiting COX-2 in an organism. In particular, the method is preferably directed toward selectively inhibiting COX-2 in an organism. The method comprises the step of administering to the organism an organic extract isolated from an edible plant wherein such extract inhibits COX-2. A method to purify a composition that exhibits COX-2 inhibition and COX-2 selective inhibition from the organic extract is also provided. In addition, a method for treating and/or preventing COX-2 mediated inflammation or inflammation-associated disorders in an organism is provided.

### CROSS-REFERENCE TO RELATED APPLICATIONS

This application is a continuation-in-part of and claims priority from U.S. application Ser. No. 09/272,363, filed Mar. 19, 1999, which is hereby incorporated by reference in its entirety.

### FIELD OF THE INVENTION

The current invention is generally directed toward nutraceuticals that are nonsteroidal anti-inflammatory agents capable of inhibiting cyclooxygenase-2 (COX-2). The present invention relates to a method for inhibition of COX-2, or selective inhibition of COX-2, in an organism by administering to the organism organic extracts isolated from edible plants wherein such extracts inhibit COX-2 activity. The present invention also relates to purified compositions of the edible plant organic extracts In addition, the current invention is directed toward a method for treating and/or preventing COX-2 mediated inflammation or inflammation-associated disorders in an organism.

### BACKGROUND OF THE INVENTION

The prostaglandins are a potent class of biologically active lipid derivatives that play a crucial role in the inflammatory response. The inflammatory response is a localized tissue response to injury or other trauma characterized by pain, heat, redness and swelling. Prostaglandins mediate this response by inhibiting platelet aggregation, increasing vascular permeability, increasing vascular dilation, inducing smooth-muscle contraction and causing the induction of neutrophil chemotaxis. Because of their central role in mediating the inflammatory response, significant efforts have been directed toward elucidating compositions that are capable of inhibiting the biosynthesis of prostaglandins.

Toward that end, prostaglandin biosynthesis has been extensively characterized. Prostaglandins are a group of oxygenated fatty acids that are generally derived from arachidonic acid . The biosynthesis of prostaglandins from arachidonic acid occurs in a three step process that includes 1) hydrolysis of arachidonic acid from phospholipid precursors catalyzed by a phospholipase A₂; 2) cyclooxygenase (“COX”) catalyzed oxygenation of arachidonic acid to prostaglandin G2 (“PGG2”). This COX catalyzed reaction is the first committed and rate limiting step in prostaglandin synthesis; and 3) conversion of prostaglandin G2 to the biologically active end product, prostaglandin, catalyzed by a series of synthases and reductases. Upon their synthesis, prostaglandins exit the cell and act in a hormone-like manner by effecting the target cell via G protein linked membrane receptors.

Inactivation of the COX enzyme is a natural target as a means to inhibit prostaglandin production due to this enzyme's pivotal role in the prostaglandin biosynthetic pathway. It is now known that two gene products possessing COX enzyme activity are expressed, termed COX-1 and COX-2. COX-1 was the first discovered isoform and is constitutively expressed in most tissue types. Because it is constitutively expressed, COX-1 is available to participate in activities requiring a rapid physiological response and causes the production of prostaglandins involved in “house-keeping” functions. For example, COX-1 is responsible for acute production of prostaglandins that regulate vascular homeostasis, maintain gastrointestinal integrity, and maintain kidney function. Thus, COX-1 activity is responsible for the synthesis of prostaglandins required for the maintenance of several cell types.

COX-2, on the other hand, is a recently discovered isoform that is inducibly expressed in response to numerous stimuli such as bacterial lipopolysaccharides, growth factors, cytokines, and phorbol esters. In addition, COX-2 is only expressed in a limited number of cell types including monocytes, macrophages, neutrophils, fibroblasts and endothelial cells. COX-2 expression, unlike COX-1 expression, has been shown to increase in rheumatoid synovial tissue. Contrastingly, COX-2 expression is inhibited in response to glucocorticoids and by anti-inflammatory cytokines. Thus, based upon these observations, COX-2 has been shown to be the isoform responsible for mediating the production of prostaglandins that participate in the inflammatory response and inflammatory related disorders. In addition, COX-2 has also been shown to participate in certain cancers, Alzheimer's disease, atherosclerosis, and central nervous system damage resulting from stroke, ischemia and trauma.

Corticosteroids provide one means to reduce effects associated with the inflammatory response. These potent anti-inflammatory agents exert their effect by causing a reduction in the number and activity of immune system cells via various mechanisms. However, prolonged administration of corticosteroids results in drastic side effects that limit the therapeutic value of this class of anti-inflammatory agent.

Nonsteroidal anti-inflammatory agents (NSAIDs) are also utilized as a means to reduce effects associated with the inflammatory response. The principal pharmaceutical effects of NSAIDs are due to their ability to prevent COX activity resulting in the inhibition of prostaglandin synthesis. Inhibition of prostaglandin synthesis by NSAIDs is anti-pyretic, analgesic, anti-inflammatory, and anti-thrombogenic. However, administration of NSAIDs may also result in severe side effects such as gastrointestinal bleeding, ulcers and incidence of renal problems. NSAIDs also inhibit both COX isoforms to varying degrees. For example, the most common NSAID, aspirin (acetylated derivative of salicylic acid), inhibits prostaglandin biosynthesis by irreversibly inactivating both COX-1 and COX-2 via acetylation of a serine residue located in the arachidonic acid binding domain. While aspirin inactivates both isoforms, it is 10 to 100 times more effective inactivating COX-1 as opposed to COX-2.

The selective inhibition of COX-2 has been shown to be anti-inflammatory and analgesic without the associated gastric and kidney related toxicity problems. This phenomenon is due to the discovery of NSAIDs that are capable of inhibiting COX-2, which is responsible for the production of prostaglandins that mediate the inflammatory response, without causing the inhibition of COX-1, which is responsible for the production of prostaglandins that maintain both gastrointestinal integrity, and kidney function. Thus, the beneficial effects of NSAIDs are separable from their drastic side effects by the development of COX-2 selective inhibitors.

Toward that end, several drugs that are COX-2 selective inhibitors of prostaglandin synthesis have been developed. The most extensively characterized class of COX-2 selective inhibitor is diarylheterocycles, which include the recently approved drugs celecoxib and rofecoxib. However, other classes include, but are not limited to, acidic sulfonamides, indomethacin analogs, zomepirac analogs, and di-t-butylphenols. For example, U.S. Pat. No. 5,380,738 describes oxazoles which selectively inhibit COX-2, U.S. Pat. No. 5,344,991 describes cyclopentenes which selectively inhibit COX-2, U.S. Pat. No. 5,393,790 describes spiro compounds which selectively inhibit COX-2, WO94/15932 describes thiophene and furan derivatives which selectively inhibit COX-2, and WO95/15316 describes pyrazolyl sulfonamide derivatives which selectively inhibit COX-2.

In order to afford an alternative to drug-based selective COX-2 therapy, it would be highly beneficial to provide nutraceuticals that inhibit COX-2, or even more preferably selectively inhibit COX-2. A nutraceutical, in this context, is an edible food or extracts therefrom that exhibit COX-2 inhibitory activity. In particular, it would be highly beneficial to obtain such edible food or extract from a plant source due to the ability to derive a large quantity of edible food or extract from a plant at a relatively affordable cost. These nutraceutical agents could be utilized in the diet in a preventative manner to maintain a “healthy” physiological state. The nutraceutical agents could also be used as a means to treat, cure or mitigate an existing inflammatory-related ailment either alone or in combination with another compound as a part of combination therapy.

### SUMMARY OF THE INVENTION

Among the several aspects of the invention therefore, is provided a method for selective inhibition of COX-2 in an organism, the method comprising the step of administering to the organism a therapeutically or prophylactically effective amount of an organic extract of an edible plant, wherein the inhibitory effect of the extract on COX-2 activity is greater than or equal to about 2 times greater than the inhibitory effect of the extract on COX-1 activity.

Another aspect of the invention is a method for inhibiting the activity of COX-2 in an organism, the method comprising the step of administering to the organism a therapeutically or prophylactically effective amount of an organic extract of an edible plant, wherein the plant is selected from the order consisting of Agavales, Apocynales, Arales, Aristolochiales, Asterales, Brassicales, Cactales, Caryophyllales, Cucurbitales, Elaeagnales, Fagales, Gnetales, Graminales, Lamiales, Liliales, Malvales, Musales, Myrtales, Papaverales, Plantaginales, Polemoniales, Ranales, Rosales, Rubiales, Rutales, Scrophulariales, Umbellales, Urticales, and Violales.

Still further is provided a method for selective inhibition of COX-2 in an organism, the method comprising the step of administering to the organism a therapeutically or prophylactically effective amount of an organic extract of an edible plant, wherein the inhibitory effect of the extract on COX-2 activity is greater than or equal to about 2 times greater than the inhibitory effect of the extract on COX-1 activity, wherein the organic extract is a purified composition obtained by a method comprising contacting the plant with an organic solvent to remove an extract from the plant wherein the extract inhibits COX-2 activity and then isolating the extract with COX-2 inhibitory activity.

In yet another aspect of the invention is provided a method of treating or preventing COX-2 mediated inflammation or an inflammation-associated disorder in an organism, the method comprising administering to the organism a therapeutically or prophylactically effective amount of a purified composition of an organic extract isolated from an edible plant wherein the purified composition is obtained by a method comprising contacting the plant with an organic solvent to remove an extract from the plant wherein the extract inhibits COX-2 activity and then isolating the extract with COX-2 inhibitory activity.

Other features of the present invention will be in part apparent to those skilled in the art and in part pointed out in the detailed description provided below.

### BRIEF DESCRIPTION OF THE DRAWINGS

These and other features, aspects, and advantages of the present invention will become better understood with regard to the following description, appended claims and accompanying figures where:

FIG. 1 depicts COX-2>COX-1 inhibition by extract isolated from Vitex agnus-castus.

FIG. 2 depicts COX-2>COX-1 inhibition by extract isolated from Citrus limonia.

FIG. 3 depicts COX-2>COX-1 inhibition by extract isolated from Citrus sp.

FIG. 4 depicts COX-2>COX-1 inhibition by extract isolated from Papaver somniferum

FIG. 5 depicts COX-2>COX-1 inhibition by extract isolated from Morus alba

FIG. 6 depicts COX-2>COX-1 inhibition by extract isolated from Abutilon sp.

FIG. 7 depicts COX-2>COX-1 inhibition by extract isolated from Coix lacryma.

FIG. 8 depicts COX-2>COX-1 inhibition by extract isolated from Artemisia dracunculus.

FIG. 9 depicts COX-2>COX-1 inhibition by extract isolated from Yucca elephantipes.

FIG. 10 depicts COX-2>COX-1 inhibition by extract isolated from Rumex japonicus.

FIG. 11 depicts COX-2>COX-1 inhibition by extract isolated from Dioscorea minutiflora.

FIG. 12 depicts COX-2>COX-1 inhibition by extract isolated from Capsicum annuum.

FIG. 13 depicts COX-2>COX-1 inhibition by extract isolated from Cissampelos mucronata.

FIG. 14 depicts COX-2>COX-1 inhibition by extract isolated from Cichorium endivia.

FIG. 15 depicts COX-2>COX-1 inhibition by extract isolated from Aster sp.

FIG. 16 depicts COX-2>COX-1 inhibition by extract isolated from Maranta arundinacea.

FIG. 17 depicts COX-2>COX-1 inhibition by extract isolated from Cynomorium sangaricum.

FIG. 18 depicts COX-2>COX-1 inhibition by extract isolated from Solanum tuberosum.

FIG. 19 depicts COX-2>COX-1 inhibition by extract isolated from Salvia sp.

FIG. 20 depicts COX-2>COX-1 inhibition by extract isolated from Stellaria media.

FIG. 21 depicts COX-2>COX-1 inhibition by extract isolated from Peucedanum sp.

FIG. 22 depicts COX-2>COX-1 inhibition by extract isolated from Asperula odorata.

### ABBREVIATIONS AND DEFINITIONS

To facilitate understanding of the invention, a number of terms and abbreviations as used herein are defined below:

“Purified” means partially purified and/or completely purified. Thus, a “purified composition” may be either partially purified or completely purified.

“Extract” means crude extract, purified extract, and purified composition obtained by purification of the extract.

“COX activity” means the ability of either COX isoform, COX-1 or COX-2, to catalyze the oxygenation reaction of arachidonic acid to PGG2.

“COX inhibitor or COX inhibition” means a composition, compound, agent or extract, purified or otherwise, that prevents either COX isoform, COX-1 or COX-2, from catalyzing the oxygenation reaction of arachidonic acid to PGG2 either in whole or in part.

“Selective inhibition of COX-2” means a composition, compound, agent, or extract, purified or otherwise, which selectively inhibits COX-2 activity over COX-1 activity as determined by the ratio of the percentage of COX-2 inhibition divided by the percentage of COX-1 inhibition, unless otherwise indicated herein.

“IC₅₀” means the concentration (in mol L⁻¹) that reduces a specified response to 50% of its former value. As used herein this value measures the amount of composition, agent or extract (ug extract/ml solvent) causing 50% inhibition of PGE2 production. The IC₅₀ value may be used to determine COX-2 selectivity as specifically set-forth herein.

“Plant or parts thereof” means either the whole plant, or any part of the plant such as an aerial part, fruit, leaf, stem, or root and any combination thereof.

“Order”, as utilized herein, is a taxonomic category of related organisms with a category consisting of a number of similar families.

“Family”, as utilized herein, is a taxonomic category of related organisms ranking below the order and above the genus.

“Species”, as utilized herein, is a fundamental taxonomic category ranking below a genus and consisting of a group of closely related individuals.

COX=the enzyme cyclooxygenase

COX-1=the isoform cyclooxygenase-1

COX-2=the isoform cyclooxygenase-2

NSAIDs=non-steroidal anti-inflammatory drugs

PGE2=prostaglandin E2

### DESCRIPTION OF THE PREFERRED EMBODIMENT

Applicants have discovered that organic extracts of certain edible plants or parts therefrom inhibit COX-2 activity. Applicants have also discovered that organic extracts of certain edible plants or parts therefrom selectively inhibit COX-2 activity. The inhibitory effect is selective because inhibition of COX-2 is greater than inhibition of COX-1. Consequently, organic extracts of the edible plants or parts therefrom may be used to selectively inhibit the activity of COX-2 in an organism without causing an equivalent inhibition of COX-1 activity. Advantageously, these organic extracts are nutraceuticals that may be safely consumed and provide an alternative to traditional drug-based therapy for COX-2 inhibition.

Accordingly, the organic extracts of the present invention preferably inhibit COX-2 activity more than COX-1 activity. Preferably, the inhibitory effect of the plant extract on COX-2 is at least about two times greater than its inhibitory effect on COX-1. In a particularly preferred embodiment, the inhibitory effect on COX-2 is at least about 10 times greater than the inhibitory effect on COX-1. COX enzyme inhibition and selectivity may be determined in accordance with any method generally known to those of ordinary skill in the field, as set forth in more detail below.

In addition to inhibiting COX-2, the organic extracts of the present invention are preferably isolated from an edible plant. As utilized herein, the term “edible” shall generally mean a substance consumed for the purpose of nourishment consisting of protein, carbohydrate (fiber or otherwise), fat and/or combinations thereof used in the body of an organism to sustain growth, repair and vital processes and to furnish energy. Classification of plants as edible versus non-edible, in addition to this general definition, is also based upon three primary criteria: (1) frequency of use as an edible substance; (2) availability in public commerce; and (3) toxicity limits due to potency. Therefore, the edible plant is preferably available to consumers in the region where the plant is provided in some form by lawful commerce. In addition, the edible plant preferably has a history of use which demonstrates that it may be safely consumed on a daily basis in amounts commonly employed in the indigenous culture where the edible plant is found for nourishment purposes. For example, a particular plant may be considered medicinal instead of edible if the plant is consumed by mouth for the purpose of correcting symptoms of illness (as opposed to nourishment) and is considered too potent to be consumed on a daily basis. Examples of edible plant uses include, but are not limited to: sources of starch, fruits, vegetables, spices, condiments, edible oils from plants, food coloring and other food additives, beverages, teas and tonics, sugar and other natural sweeteners, fermented beverages, ferments and enzymes, non-narcotic chewing leaves and gums, woody flavorings, and all other natural substances which are eaten or imbibed regularly to maintain health, sustain growth, repair injuries, and promote general well-being. In addition, any plant classified as edible by those of general skill in the art is included in the scope of the present invention, for example, such references include, NAPRALERT; Tyozaburo Tanaka, (Edited by Sasuke Nakoa) Tanaka's Cyclopedia of Edible Plants of the World, Keigaku Publishing Co., Tokyo, Japan, 1976; Stephen Facciola, Cornucopia II: A Source Book of Edible Plants, Kampong Publications, Vista, Calif., 1998; James A. Duke, Database of Phytochemical constituents of GRAS Herbs and Other Economic Plants, CRC Press, Boca Raton, Fla., 1992; and George Macdonald Hocking, Dictionary of Natural Products, Plexus Publishing, Inc., Medford, N.J., 1997. The contents of these references are hereby incorporated in their entirety.

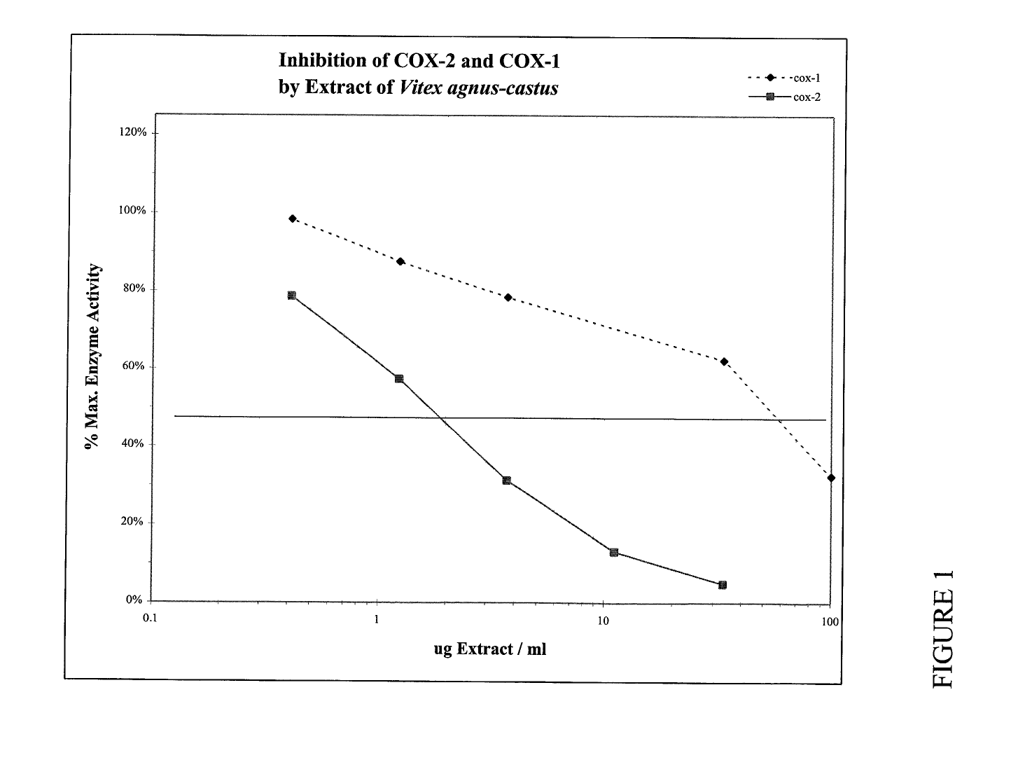

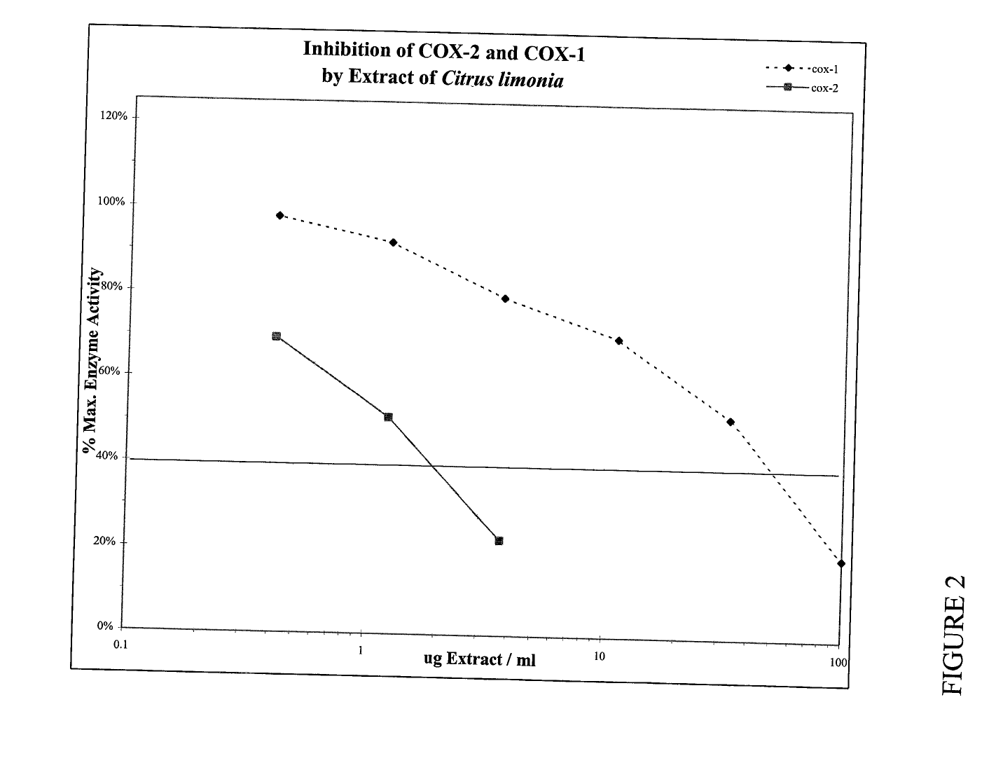

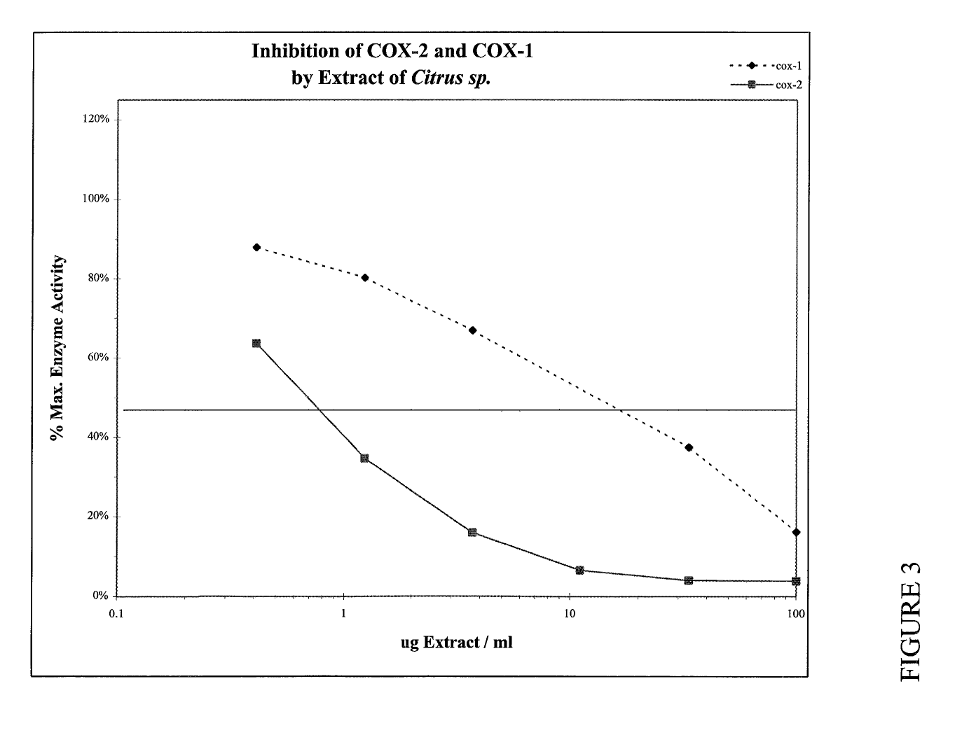

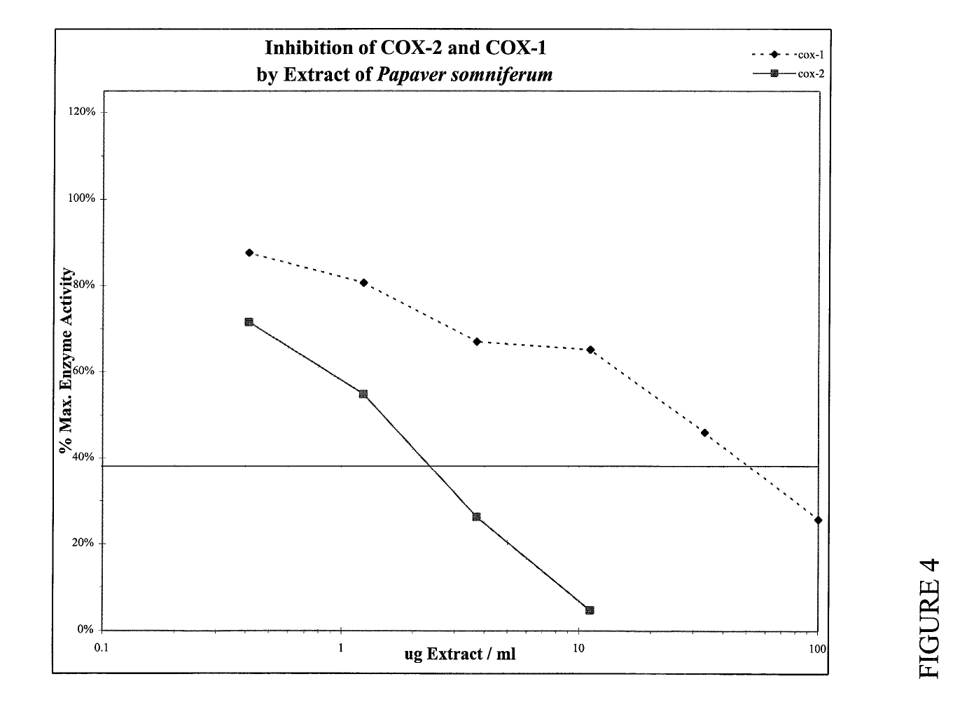

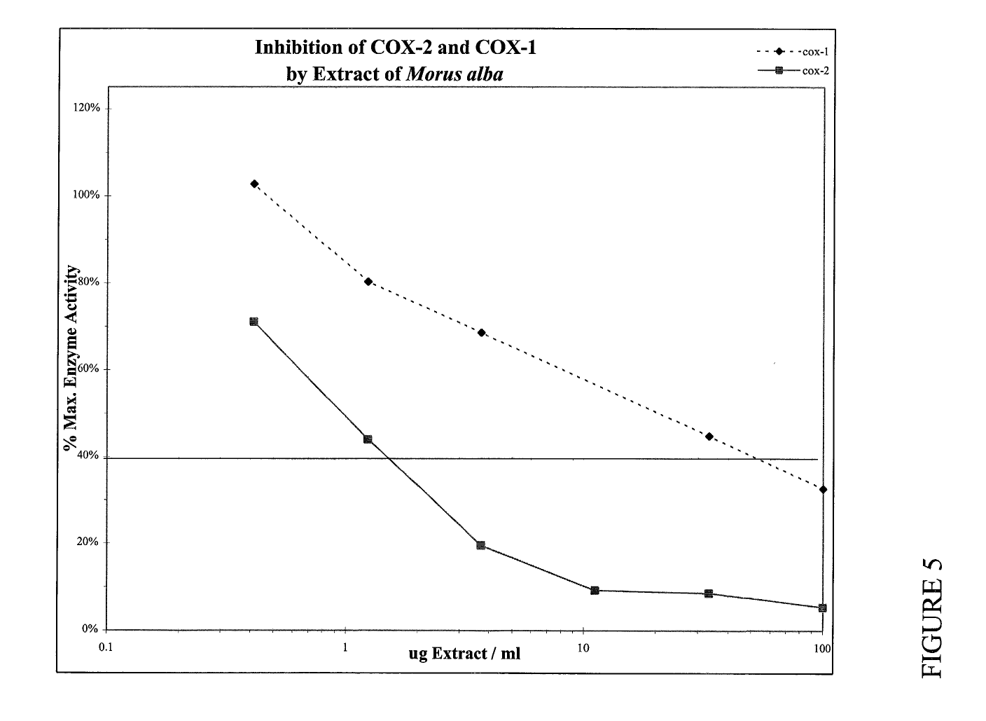

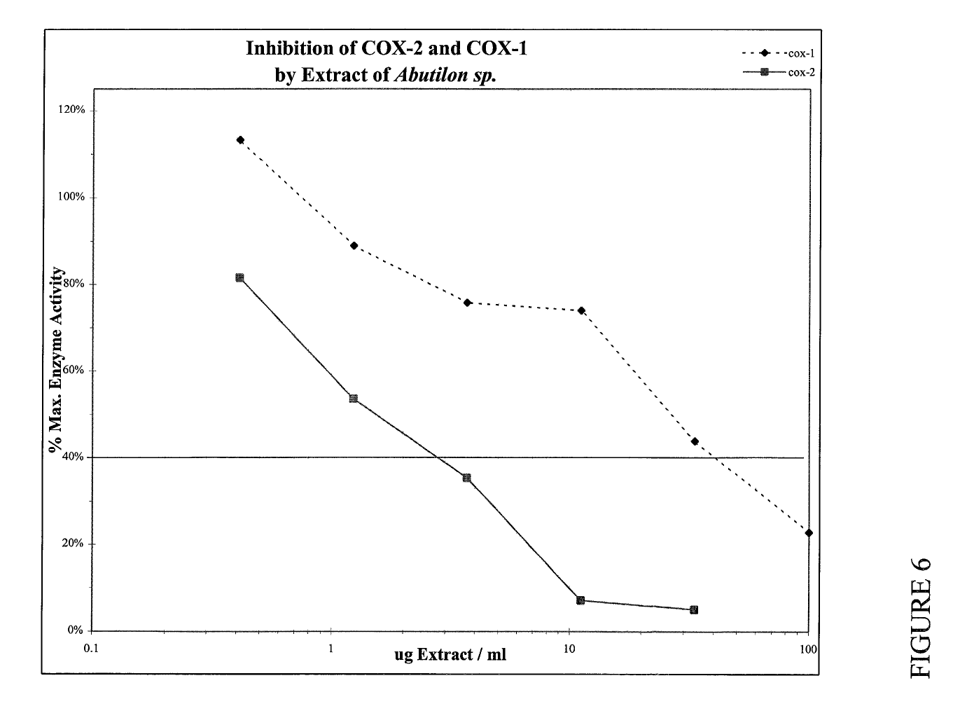

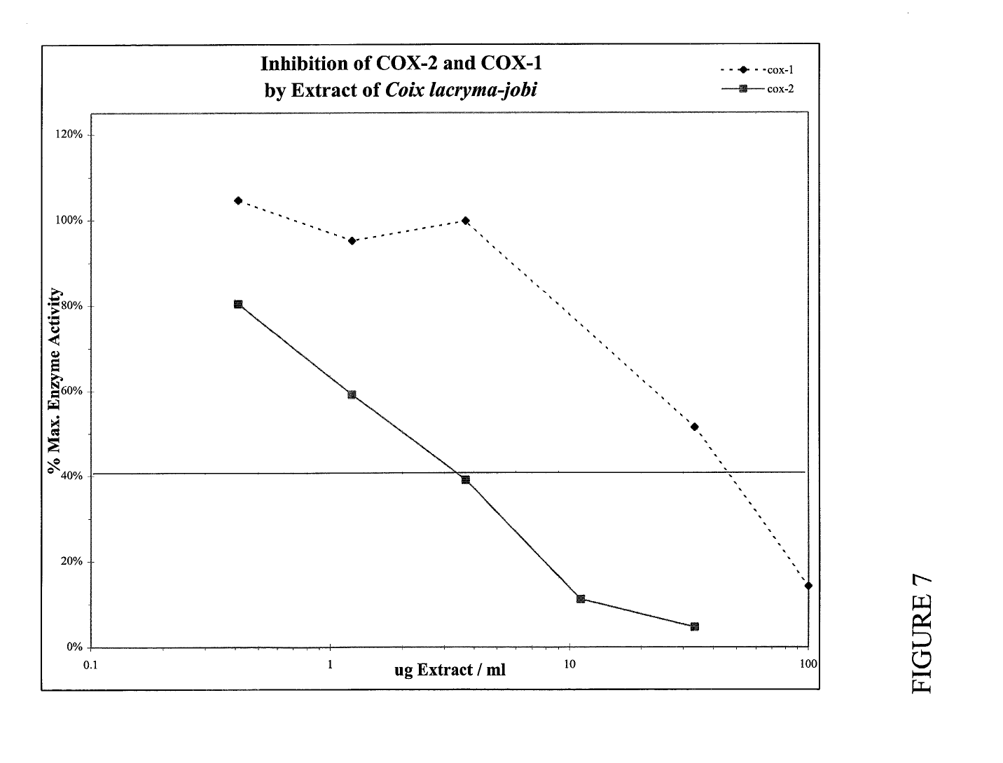

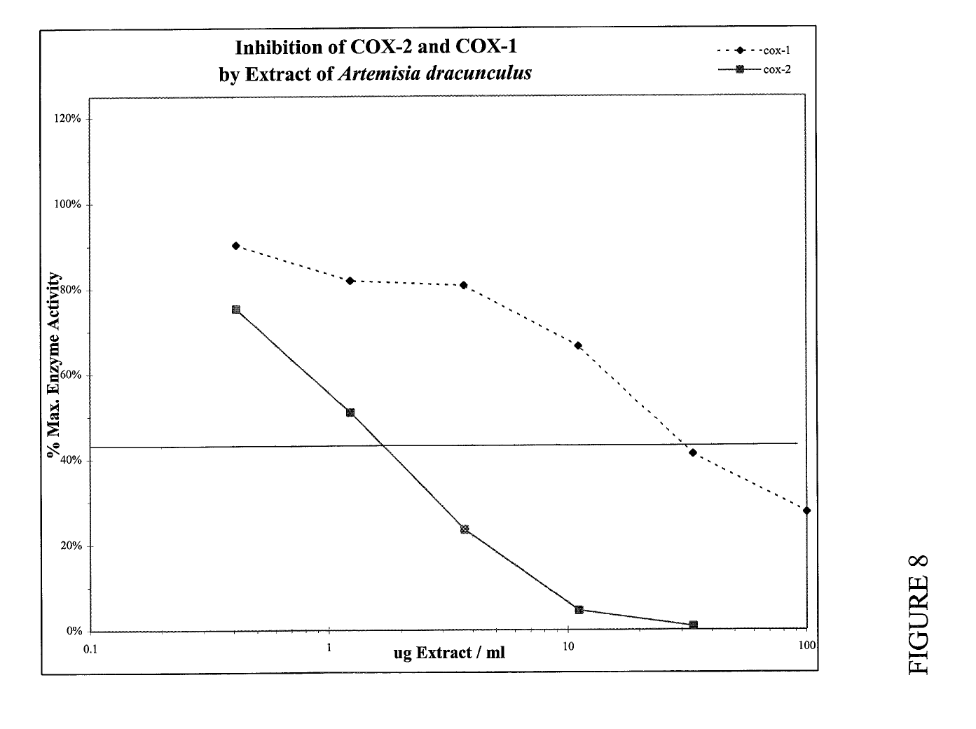

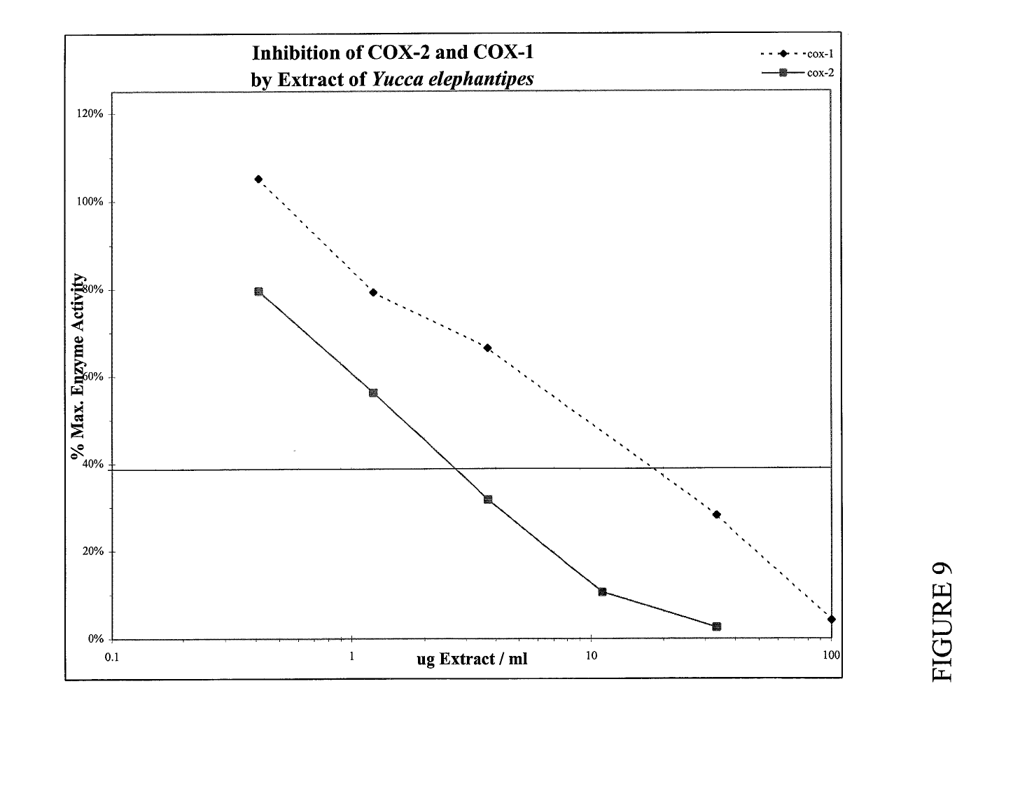

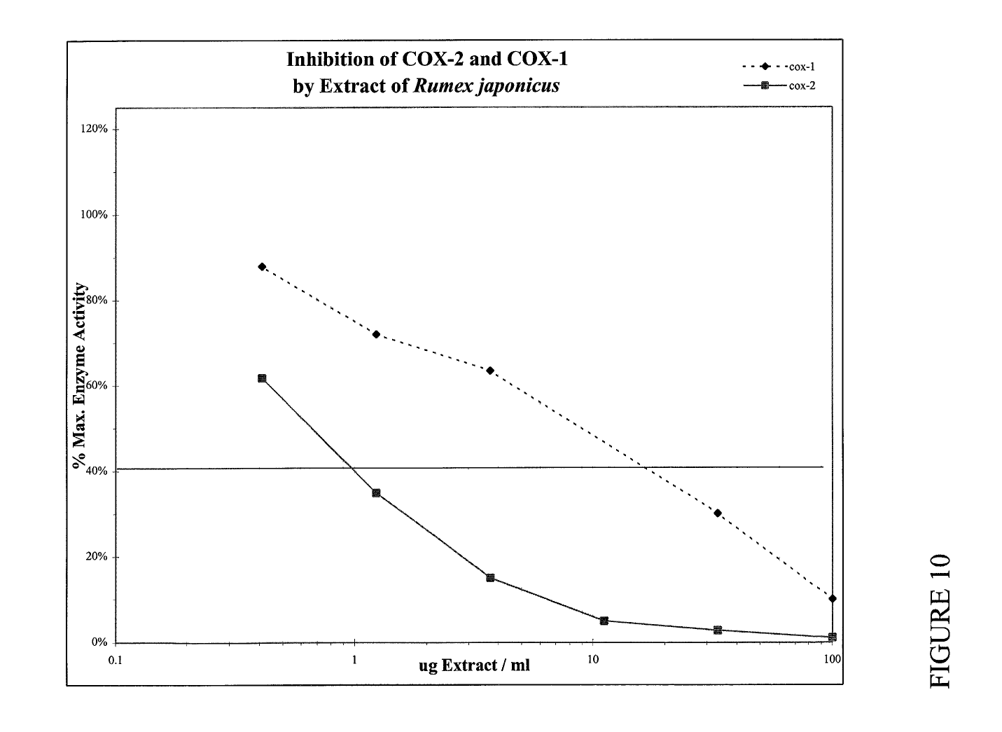

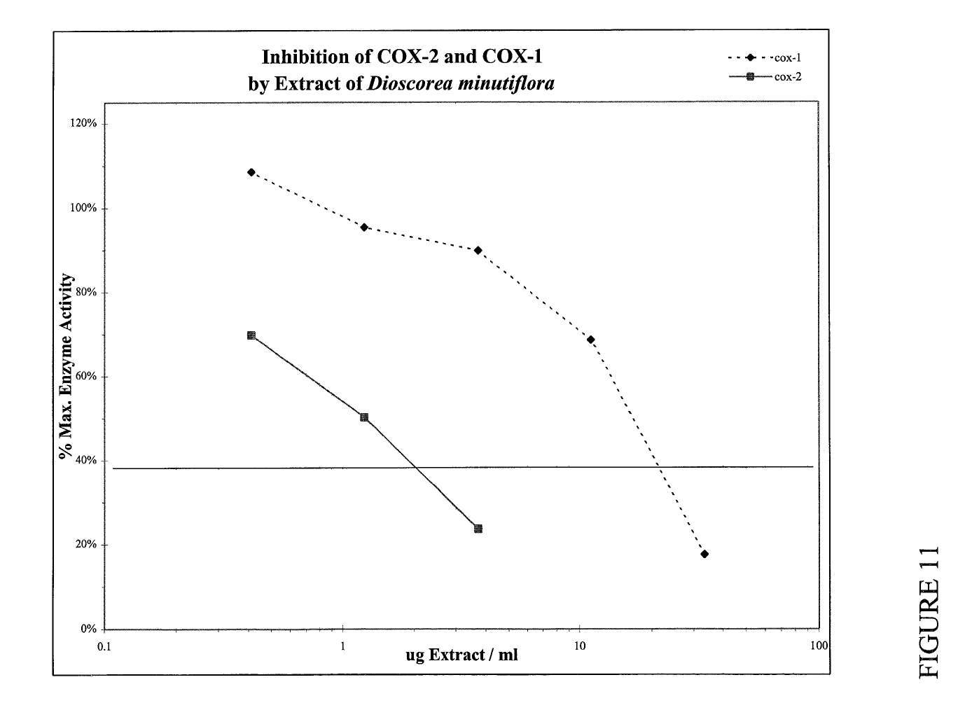

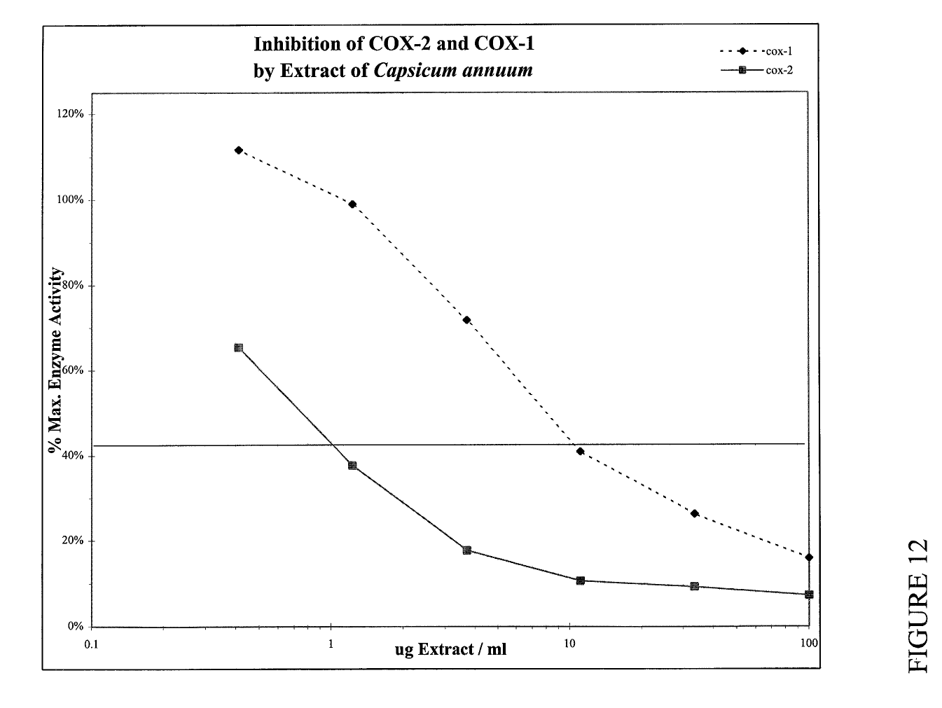

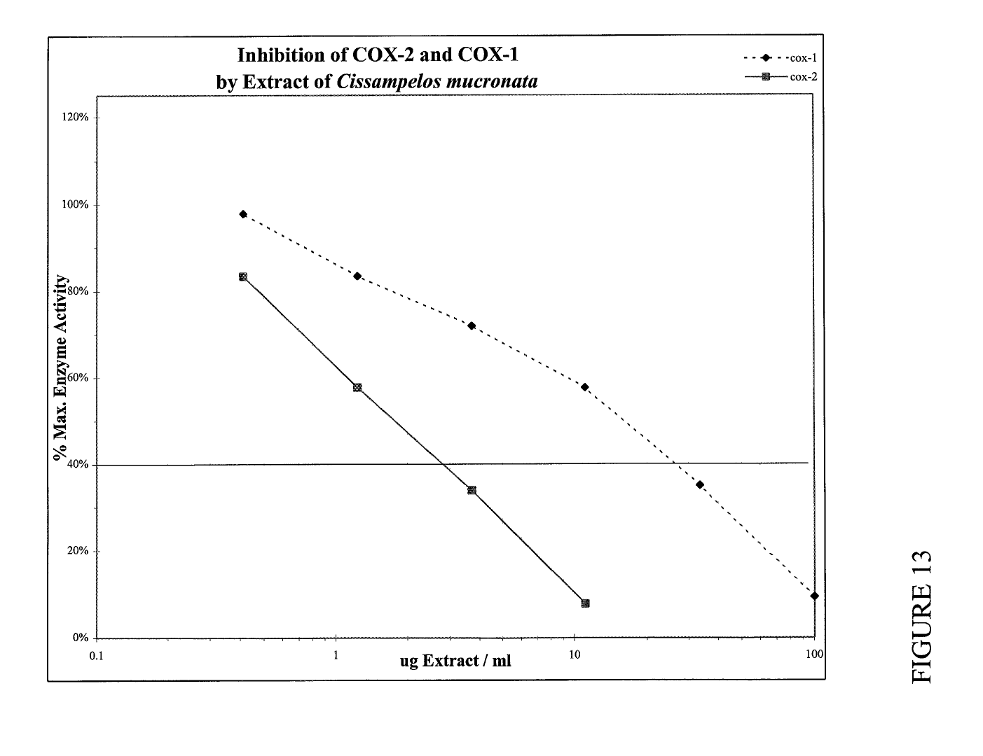

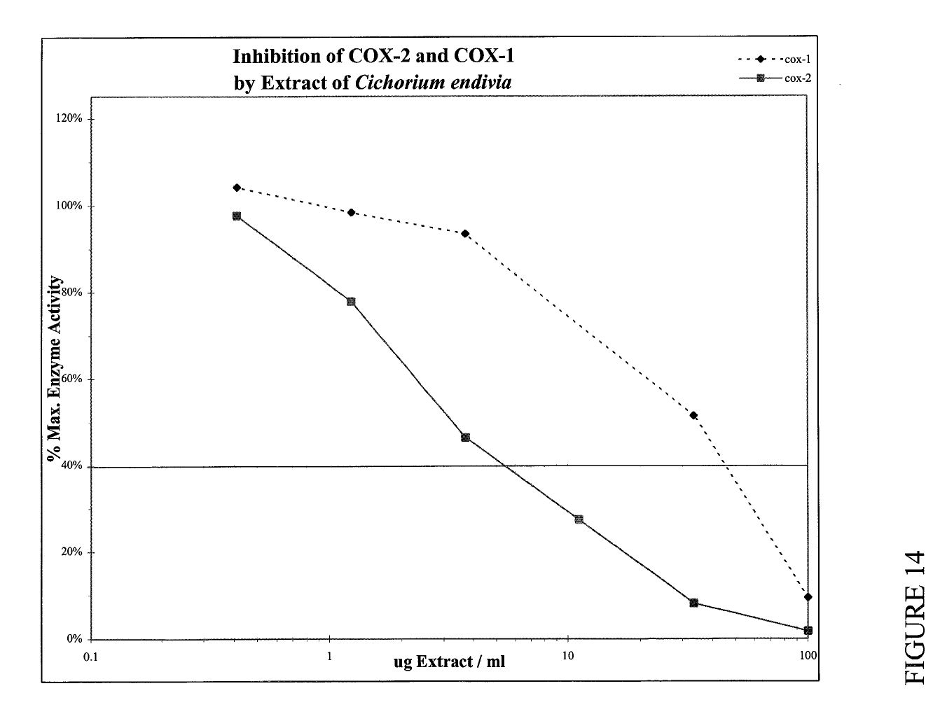

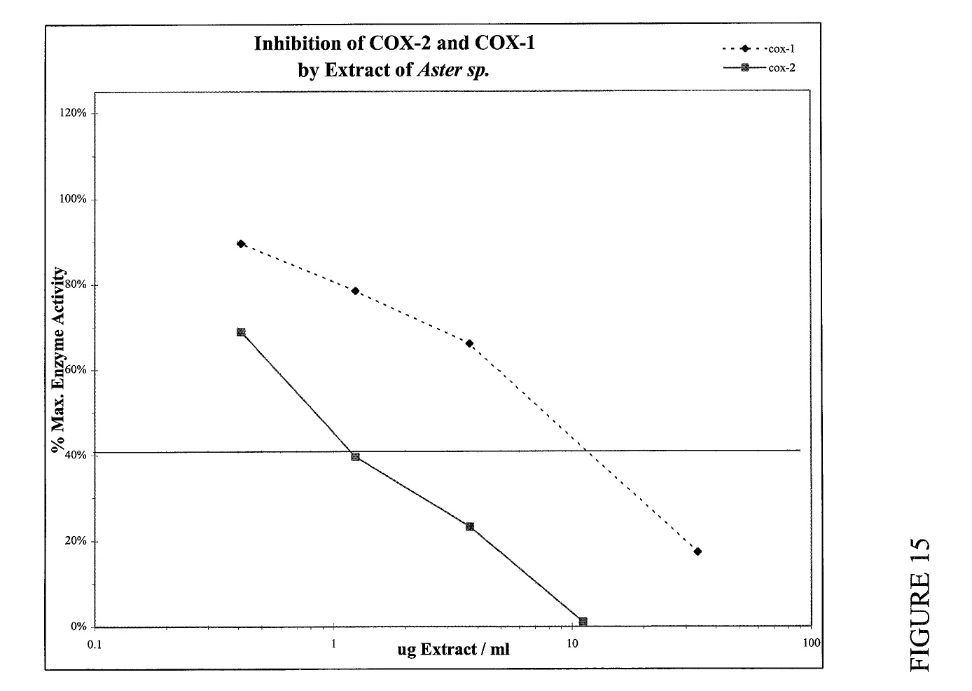

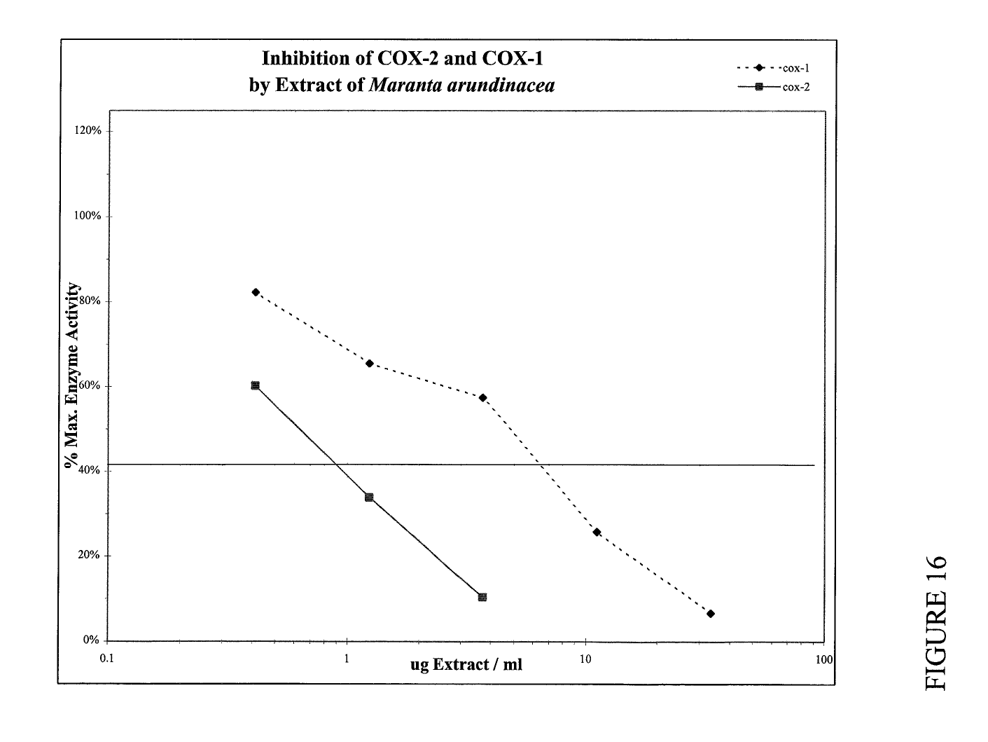

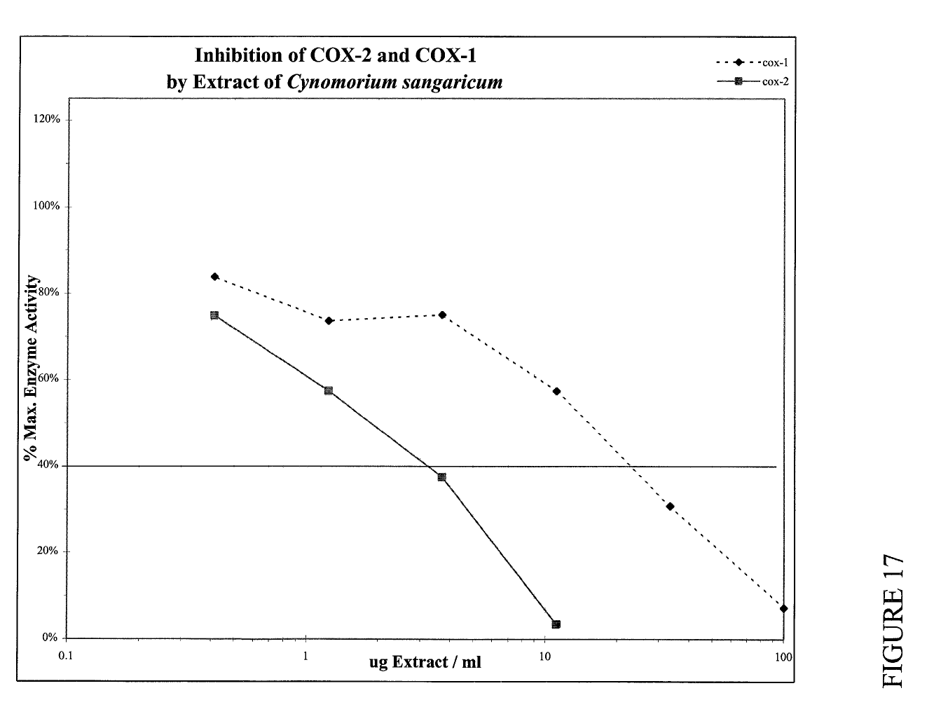

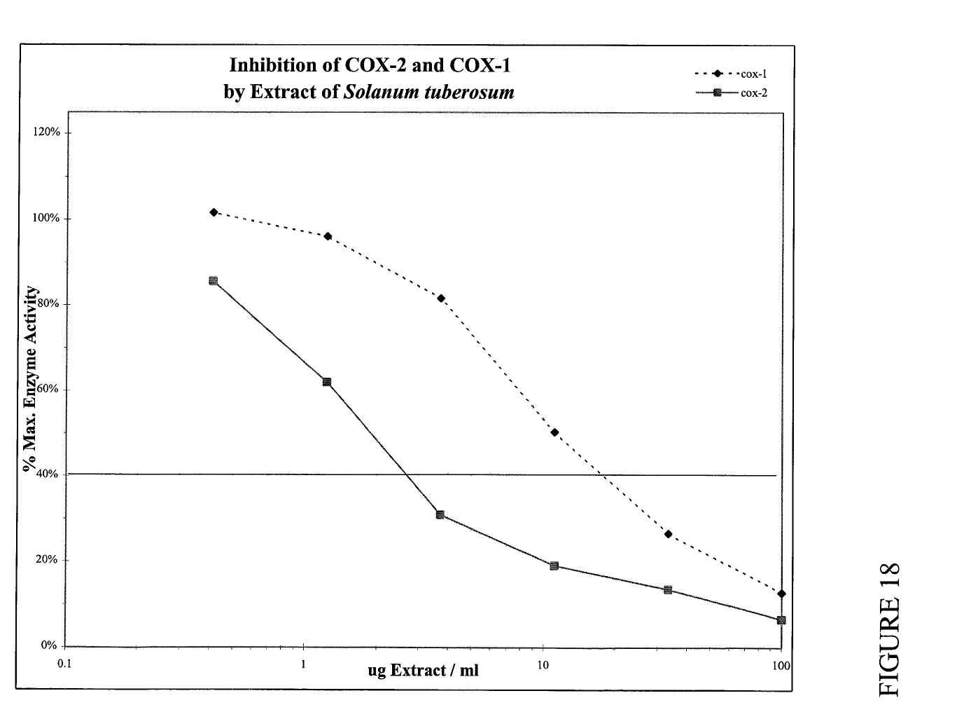

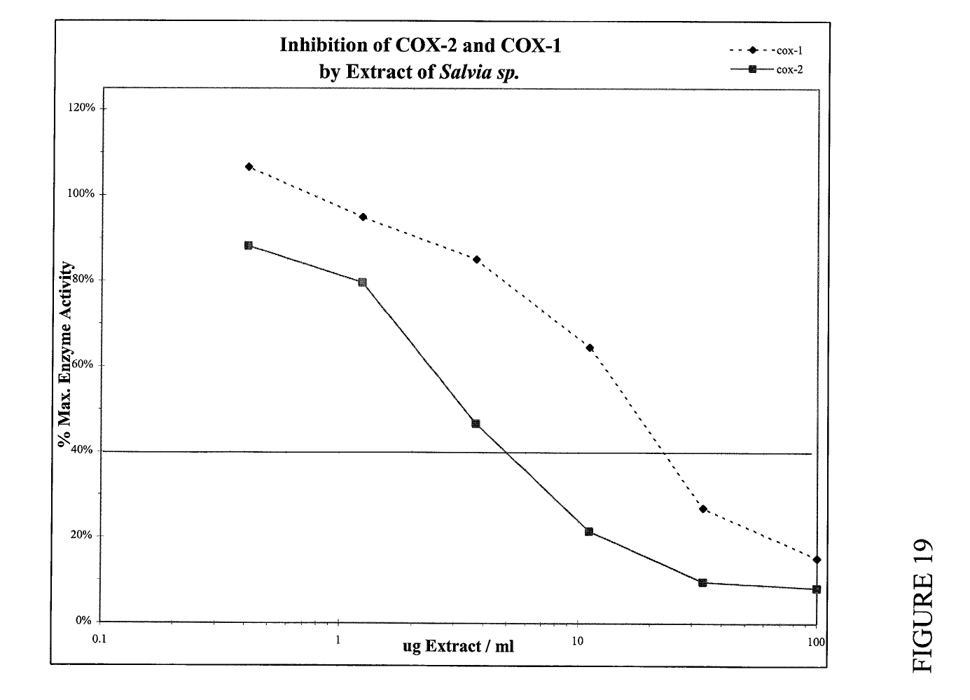

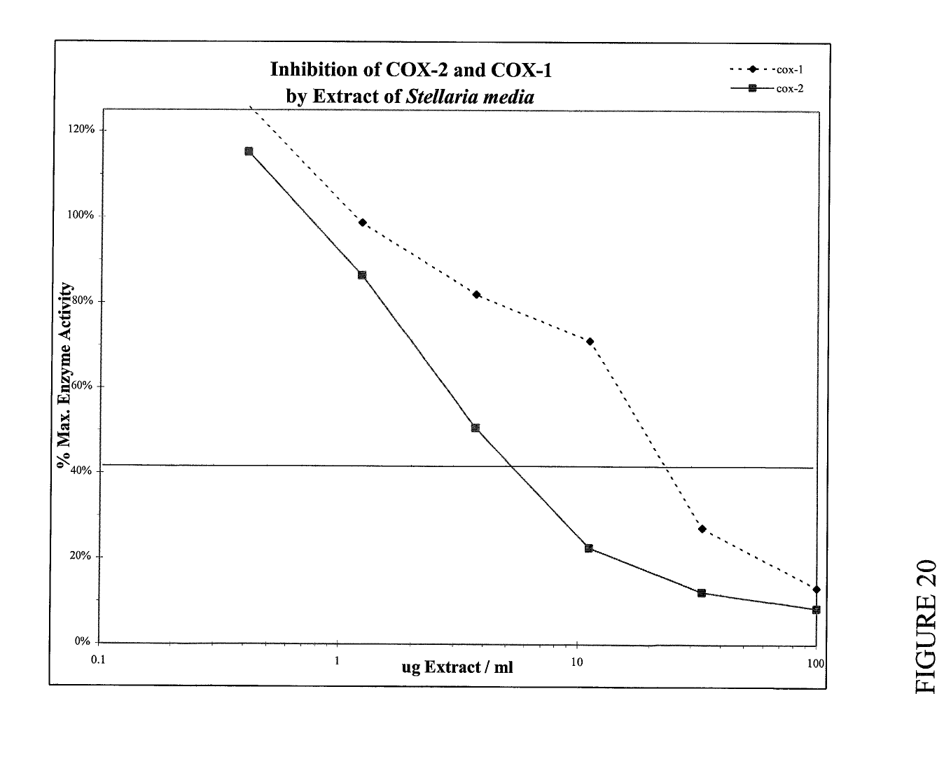

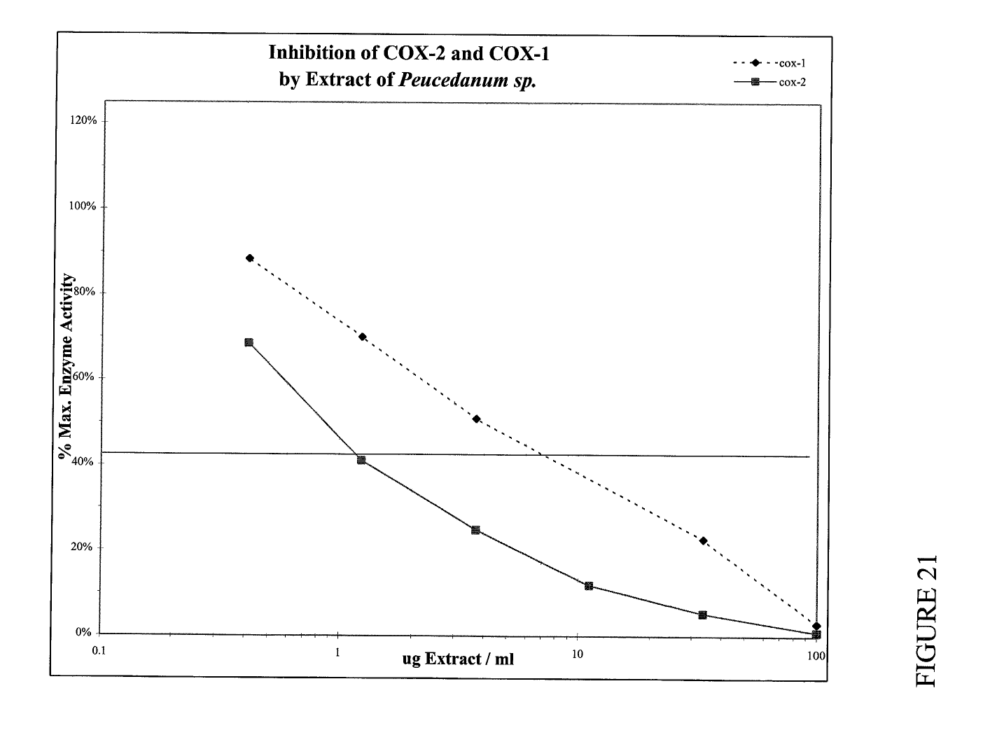

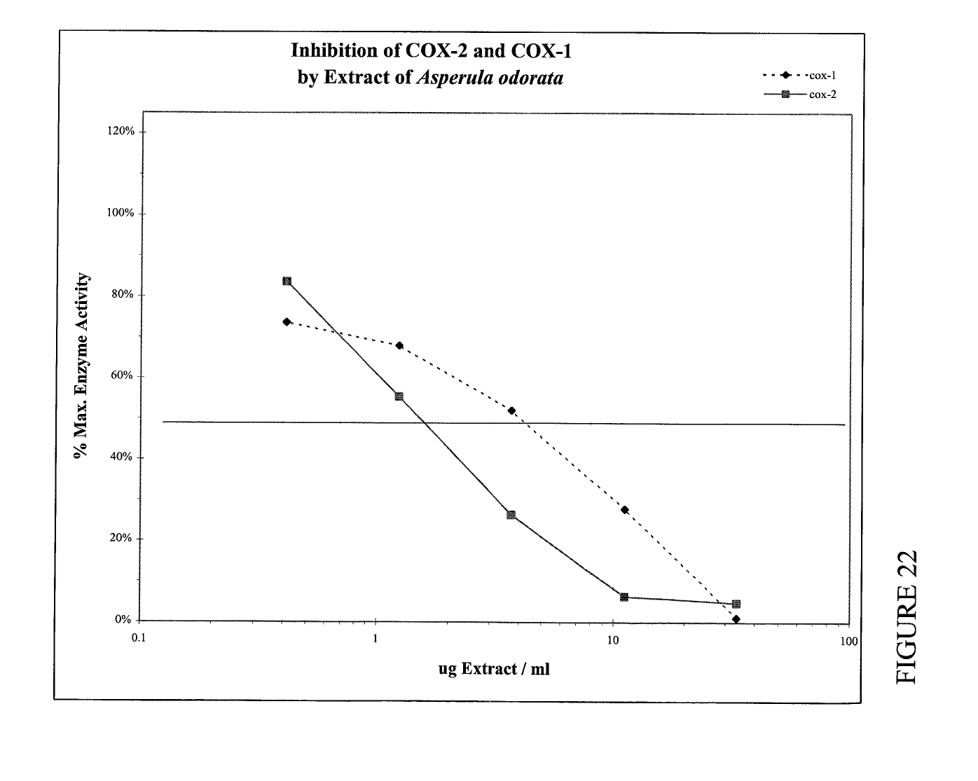

In a particularly preferred embodiment, organic extracts are isolated from edible plants of the following plant orders: Agavales, Apocynales, Arales, Aristolochiales, Asterales, Brassicales, Cactales, Caryophyllales, Cucurbitales, Elaeagnales, Fagales, Gnetales, Graminales, Lamiales, Liliales, Malvales, Musales, Myrtales, Papaverales, Plantaginales, Polemoniales, Ranales, Rosales, Rubiales, Rutales, Scrophulariales, Umbellales, Urticales, and Violales. The ability of extracts isolated from edible plants of these particular orders to inhibit COX-2, to selectively inhibit COX-2, and their use as edible plants are set-forth below in Tables 1-24 and FIGS. 1-22.

It is to be understood that while applicant contemplates as within his invention the use of any organic extract isolated from edible plants wherein such extract inhibits COX-2 activity and preferably, wherein the inhibitory effect of such extract on COX-2 activity is greater than or equal to about 2 times greater than the inhibitory effect of the extract on COX-1 activity, that also included within applicant's contemplation are the use of such class or classes, but excluding any particular member(s) (e.g., species, genus or order) which may be previously disclosed and used and which inherently or otherwise possesses such required activity. For example, applicant's invention herein may include or exclude as appropriate, the full scope of the invention as related to Atractylodes lancea as set forth in applicant's U.S. application Ser. No. 09/272,363, which is fully incorporated herein by reference.

In order to prepare the organic extracts of the invention, an edible plant or parts thereof are preferably ground into a fine powder, the resultant powder is extracted with a solvent, and the extraction solvent is removed from the extract. The whole plant may be used or parts of the plant including an aerial part, fruit, leaf, stem, or root and any combination thereof may be utilized. If desired, the resultant extract may be further purified to yield a purified extract or one or more purified compositions. The grinding step may be accomplished by any commonly known method for grinding a plant substance. For example, the plant or parts thereof may be passed through a grinder to obtain a fine powder.

After the plant or parts thereof have been ground into a fine powder, they are combined with an extraction solvent. The solution is then stirred at a temperature, and for a period of time, that is effective to obtain an extract with the desired inhibitory effects on the activity of COX-2. The solution is preferably not overheated, as this may result in degradation and/or denaturation of compounds in the extract. The solution may be stirred at a temperature between about room temperature (25° C.) and the boiling point of the extraction solvent. Preferably, the solution is stirred at about room temperature.

The length of time during which the plant powder is exposed to the extraction solvent is not critical. Up to a point, the longer the plant powder is exposed to the extraction solvent, the greater is the amount of extract that may be recovered. Preferably, the solution is stirred for at least 1 minute, more preferably for at least 15 minutes, and most preferably for at least 60 minutes.

The extraction process of the present invention is desirably carried out using an organic solvent or a mixture of organic solvents. Organic solvents which may be used in the extraction process of the present invention, include but are not limited to hydrocarbon solvents, ether solvents, chlorinated solvents, acetone, ethyl acetate, butanol, ethanol, methanol, isopropyl alcohol and mixtures thereof. Hydrocarbon solvents which may be used in the present invention include heptane, hexane and pentane. Ether solvents which may be used in the present invention include diethyl ether. Chlorinated solvents which may be used in the present invention include dichloromethane and chloroform. Preferably, the solvent utilized for such extraction is a nonpolar organic solvent, such as dichloromethane or hexane.

The relative amount of solvent used in the extraction process may vary considerably, depending upon the particular solvent employed. Typically, for each 100 grams of plant powder to be extracted, about 500 ml of extraction solvent would be used. The organic solvent may be removed from the extract by any method known in the field of chemistry for removing organic solvents from a desired product, including, for example, rotary evaporation.

It is believed that the inhibitory effect of the plant extract of this invention on the activity of COX-2 is due to the presence of one or more compounds in the extract. Compounds present in the extract which inhibit the activity of COX-2 may be isolated and purified by those of ordinary skill in the art employing methods known in the art. For example, column chromatography and fractional distillation may be used to obtain pure compounds from the plant extract of this invention.

The isolation and purification of particular compounds from the organic plant extracts of this invention may be performed as described in Resch, et al., J. Nat. Prod., 61, 347-350 (1998), the entire contents of which are incorporated by reference herein. The methods disclosed therein may be used to isolate and purify compositions which inhibit COX-2.

The ability of a particular organic extract to inhibit COX-1 or COX-2 is preferably determined by performing COX activity assays utilizing recombinant COX-1 and COX-2. The COX-1 and COX-2 genes may be subcloned from a variety of organisms, however in a preferred embodiment such genes are isolated from human or murine sources, using a variety of procedures known to those skilled in the art and detailed in, for example, Sambrook et al., Molecular Cloning, A Laboratory Manual, 2nd ed., Cold Spring Harbor Laboratory Press, (1989) and Ausabel et al., Short Protocols in Molecular Biology, 3rd. ed., John Wiley & Sons (1995). Additionally, the subcloned portion of the particular COX gene may be inserted into a vector by a variety of methods. In a preferred method, the sequence is inserted into an appropriate restriction endonuclease site(s) in a baculovirus transfer vector pVL1393 utilizing procedures known to those skilled in the art and detailed in, for example, Sambrook et al., Molecular Cloning, A Laboratory Manual, 2nd ed., Cold Spring Harbor Laboratory Press, (1989) and Ausubel et al., Short Protocols in Molecular Biology, 3rd ed., John Wiley & Sons (1995).

The recombinant baculoviruses may be isolated by transfecting an appropriate amount of baculovirus transfer vector DNA into a sufficient quantity of SF9 insect cells along with linearized baculovirus plasmid DNA by the calcium phosphate method or any other method generally know to those skilled in the art. (See M. D. Summers and G. E. Smith, A Manual of Methods for Baculovirus Vectors and Insect Cell Culture Procedures, Texas Agric. Exp. Station Bull. 1555 (1987)). Recombinant viruses may be purified by three rounds of plaque purification and high titer (10⁷-10⁸ pfu/ml) stocks of virus may be prepared.

Preferably, for large scale production, cells may be infected in approximately 10 liter fermentors (0.5×10⁶/ ml) with the recombinant virus stock such that the multiplicity of infection is greater than about 0.1. After several hours the cells are centrifuged and the cell pellet is homogenized in an appropriate buffer such as Tris/sucrose (50 mM/25%, pH 8.0). The homogenate may then be centrifuged at an appropriate speed and for an appropriate time (such as 10,000× G for 30 minutes) so as to cause the homogenate to separate into a pellet and supernatant fraction. The resultant supernatant fraction will contain the desired product and may be stored at −80° C. until use.

In order to test organic extracts for COX-2 inhibition and selectivity, standard COX-1 and COX-2 assays may be performed by employing ELISA procedures generally known to those skilled in the art. In such procedures, COX-1 and COX-2 activities are assayed as PGE₂ formed/μg protein/time using ELISA to detect the amount of PGE₂ synthesized from arachidonic acid. PGE₂ formation may be measured using PGE₂ specific antibody. Indomethacin, a non-selective COX-2/COX-1 inhibitor, may be employed as a positive control. The relative ability of various organic extracts to inhibit COX-1 or COX-2 at a particular concentration may be determined by comparing the IC₅₀ value expressed as μg extract/ml solvent resulting in a 50% inhibition of PGE₂ production. Selective inhibition of COX-2 may then be determined by the IC₅₀ ratio of COX-1/COX-2. Additionally, any other means to determine COX inhibition known to those generally skilled in the art may be employed, for example, determining the ratio of percent inhibition of COX-1/COX-2 at a fixed concentration of test agent.

The extracts of this invention may be used to manage, prevent and/or treat an organism having, or at risk for developing, a condition which is mediated in whole or in part by COX-2. Accordingly, conditions which may be benefited by inhibition of COX-2 or selective inhibition of COX-2 include, but are not limited to, the treatment of inflammation in an organism, and for treatment of other inflammation-associated disorders, such as, an analgesic in the treatment of pain and headaches, or as an antipyretic for the treatment of fever. For example, extracts of the invention would be useful to treat arthritis, including but not limited to rheumatoid arthritis, spondyloarthopathies, gouty arthritis, osteoarthritis, systemic lupus erythematosus and juvenile arthritis. Such extracts of the invention would be useful in the treatment of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Extracts of the invention also would be useful to treat gastrointestinal conditions such as inflammatory bowel disease, Crohn's disease, gastritis, irritable bowel syndrome and ulcerative colitis, and treatment of cancer, including but not limited to the following types of cancer: colon, breast, prostate, bladder, or lung. In yet another preferred use, the extracts of the present invention may also be utilized as chemopreventive agents. Extracts of the invention would be useful in treating inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like. The extracts would also be useful in the treatment of ophthalmic diseases, such as retinitis, retinopathies, uveitis, ocular photophobia, and of acute injury to the eye tissue. The extracts would also be useful in the treatment of pulmonary inflammation, such as that associated with viral infections and cystic fibrosis. Additionally, the extracts would be beneficial for the treatment of certain central nervous system disorders such as cortical dementias including Alzheimer's disease. The extracts of the invention are useful as anti-inflammatory agents, such as for the treatment of arthritis, with the additional benefit of having significantly less harmful side effects. These extracts would also be beneficial in the treatment of allergic rhinitis, respiratory distress syndrome, endotoxin shock syndrome, atherosclerosis and central nervous system damage resulting from stroke, ischemia and trauma. Additionally, the extracts would be useful in the treatment of pain, including but not limited to postoperative pain, dental pain, muscular pain, and pain resulting from cancer.

The present extracts may also be employed either alone or in combination with other compounds as a part of combination therapy, partially or completely, in place of other conventional anti-inflammatories. For example, such as together with steroids, NSAIDs, 5-lipoxygenase inhibitors, leukotriene antagonists, LTA4 hydrolase inhibitors, and LTC4 synthase inhibitors. Preferably, with combination therapy, one will typically combine a drug or drugs and a nutraceutical, such as a plant extract of the current invention, in a manner such that the drug and the nutraceutical have different mechanisms of action, but yet target the same disease. For example, in a typical selection of agents for use in combination therapy to treat arthritis, one could utilize a plant extract of the present invention, which exhibits selective COX-2 inhibition with another agent known to attenuate inflammation associated with arthritis via an independent mechanism.

Those of ordinary skill in the art of preparing pharmaceutical formulations can readily formulate pharmaceutical compositions having plant extracts using known excipients (e.g., saline, glucose, starch, etc.). Similarly, those of ordinary skill in the art of preparing nutritional formulations can readily formulate nutritional compositions having plant extracts. And those of ordinary skill in the art of preparing food or food ingredient formulations can readily formulate food compositions or food ingredient compositions having plant extracts.

In addition, those of ordinary skill in the art can readily determine appropriate dosages that are necessary to achieve the desired therapeutic, prophylactic, pathologic or resuscitative effect upon oral, parenteral, rectal and other administration forms to the organism. Typically, in vivo models (i.e., laboratory mammals) are used to determine the appropriate plasma concentrations necessary to achieve a desired mitigation of inflammation related conditions.

The extracts of the present invention may be employed for the treatment and/or prevention of inflammation-related disorders, as identified above, in a number of organisms. Besides being useful for human treatment, these extracts are also useful for veterinary treatment of companion animals, exotic animals and farm animals, including mammals, rodents, avians, and the like. More preferred animals include horses, dogs, cats, sheep, and pigs.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Sample Preparation

Plants or parts thereof were dried and sliced (“sample”). Samples of organic extracts were prepared from the edible plants listed in Table 1. The plant orders and families that the various samples were prepared from are also set forth in Table 1. In addition, details regarding the use of these plants as edibles is set-forth in Table 2. The particular sample was then ground into a fine powder using a coffee grinder. Approximately 100 grams of the resulting powder were added to approximately 500 ml of dichloromethane and stirred at room temperature for about 1 hour. The solvent was then removed by rotary evaporation, leaving several grams of the particular extract.

### Inhibitory Effect of Various Plant Organic Extracts on COX-1 and COX-2 Activity

The particular extracts resulting from the sample preparation procedure detailed above were each evaluated for selective inhibition of COX-1 and COX-2. The COX-1 and COX-2 inhibition activities were determined in vitro according to the method of Gierse et al., J. Biochem., 305, 479-484 (1995). This method is summarized below.

Preparation of recombinant COX baculoviruses

Recombinant COX-1 was prepared by cloning a 2.0 kb fragment containing the coding region of human or murine COX-1 into a BamH1 site of the baculovirus transfer vector pVL1393 (Invitrogen) to generate the baculovirus transfer vectors for COX-1 according to the method of D. R. O'Reilly et al., Baculovirus Expression Vectors: A Laboratory Manual (1992).

Recombinant baculoviruses were then isolated by transfecting 4 μg of baculovirus transfer vector DNA into (2×10⁸) SF9 insect cells along with 200 μg of linearized baculovirus plasmid DNA by the calcium phosphate method. (See M. D. Summers and G. E. Smith, A Manual of Methods for Baculovirus Vectors and Insect Cell Culture Procedures, Texas Agric. Exp. Station Bull. 1555 (1987)). Recombinant viruses were purified by three rounds of plaque purification and high titer (10⁷-10⁸ pfu/ml) stocks of virus were prepared.

For large scale production, SF9 insect cells were infected in 10 liter fermentors (0.5×10⁶/ml) with the recombinant baculovirus stock such that the multiplicity of infection was 0.1. After 72 hours the cells were centrifuged and the cell pellet was homogenized in Tris/sucrose (50 mM/25%, pH 8.0) containing 1% of 3-[(3-cholamidopropyl)dimethylammonio]-1-propanesulfonate (CHAPS). The homogenate was then centrifuged at 10,000× G for 30 minutes, and the resultant supernatant was stored at −80° C. until use.

Recombinant COX-2 was prepared by cloning a 2.0 kb fragment containing the coding region of human or murine COX-2 in accordance with the same method described above for COX-1.

Assay for COX-1 and COX-2 Activities

COX-1 and COX-2 activities were assayed as prostaglandin E2 (PGE2) formed/μg protein/time using ELISA to detect PGE2 synthesized from arachidonic acid. CHAPS-solubilized insect cell membranes containing recombinant COX-1 or COX-2 enzyme were incubated in a potassium phosphate buffer (50 mM, pH 8.0) containing epinephrine, phenol, and heme. Compounds or extracts were pre-incubated with the appropriate enzyme for approximately 10-20 minutes. Arachidonic acid (10 M) was then added to the mixture and the reaction was permitted to occur for ten minutes at room temperature (25° C.).

Any reaction between the arachidonic acid and the enzyme was stopped after ten minutes by transferring 40 ml of reaction mixture into 160 ml ELISA buffer and 25 M indomethacin. Indomethacin, a non-selective COX-2/COX-1 inhibitor, was utilized as a positive control. The PGE₂ formed was measured by standard ELISA technology utilizing a PGE2 specific antibody (Cayman Chemical).

Approximately 200 mg of each extract obtained from the sample preparation procedure set-forth above were each individually dissolved in 2 ml of dimethyl sulfoxide (DMSO) for bioassay testing to determine the COX-1 and COX-2 inhibitory effects of each particular extract. Potency was determined by the IC₅₀ value expressed as g extract/ml solvent resulting in a 50% inhibition of PGE2 production. Selective inhibition of COX-2 was determined by the IC₅₀ ratio of COX-1/COX-2. The results of these bioassays performed utilizing extract isolated from the plant variety indicated are reported in Tables 3-24 and FIGS. 1-22 delineated below.

Table 1 below sets forth results of screening extracts of edible plants isolated from the orders, families, genera, and species indicated. A primary screen (indicated as 1° assay in Table 1) was performed in order to determine particular extracts that inhibit COX-2 at a concentration of 10 ug/ml. The extracts were then subjected to a confirmation assay to determine the extent of COX-2 inhibition at three different concentrations (10 ug/ml, 3.3 ug/ml and 1.1 ug/ml). The extracts were then tested for their ability to inhibit COX-1 at a concentration of 10 ug/ml. The percentage of COX inhibition compared to control is indicated as a percentage in each column, with a higher percentage indicating a greater degree of COX inhibition. In addition, the IC₅₀ value for COX-1 and COX-2 was also determined for certain extracts as indicated in Table 1. The selectivity for these extracts was then determined by the IC₅₀ ratio of COX-1/COX-2, as set-forth above. The COX-2 selectivity of extracts whose IC₅₀ value was not determined may be calculated by dividing the percentage of COX-2 inhibition (at a concentration of 10 ug/ml) by the percentage of COX-1 inhibition (at a concentration of 10 ug/ml).

TABLE 1
Extracts from Edible Plants that inhibit COX-2

| | | | | | | 1° assay | Confirmation assay | | | | | | |
|---|---|---|---|---|---|---|---|---|---|---|---|---|---|
| | | | | | | COX-2 (% inhib.) | COX-2 (% inhib.) | | | COX-1 (% inhib.) | IC50 (ug/ml) | IC50 (ug/ml) | Selectivity |
| Order | Family | Genus | Species | Common name | Part | 10 ug/ml | 10 ug/ml | 3.3 ug/ml | 1.1 ug/ml | 10 ug/ml | COX-2 | COX-1 | COX-2/COX-1 |
| Agavales | Agavaceae | Yucca | elephantipes | izote; Spanish dagger | | 88% | 83% | 46% | 40% | 15% | 0.7 | 10 | 14 |
| Apocynales | Asclepiadaceac | Asclepias | tuberosa | pleurisy root | | 82% | 93% | ** | 17% | 8% | *** | *** | *** |
| Arales⁹ | Araceae | Acorus | calamus | calamus root | | 76% | 78% | 57% | 64% | 39% | *** | *** | *** |
| Arales⁹ | Araceae | Acorus | gramineus | shih-chang | RT | 91% | 84% | 52% | 29% | 53% | *** | *** | *** |
| Arales⁹ | Araccac | Colocasia | esculenta | malanga coco | | 77% | 82% | 46% | 37% | 21% | *** | *** | *** |
| Arales⁹ | Araceae | Colocasia | esculenta | taro | LF | 76% | 100% | ** | 30% | 32% | *** | *** | *** |
| Arales⁹ | Araceae | Xanthosoma | sagittifolium | malanga | LF | 87% | 96% | ** | 31% | 37% | *** | *** | *** |
| Arales⁹ | Araceae | Xanthosoma | sagittifolium | malanga | PT | 76% | 94% | ** | 27% | −82% | 15 | 30 | 2 |
| Aristocholiales | Arissolochiaccac | Aristolochia | unidentified | | | 78% | 89% | 67% | 49% | 18 | *** | *** | *** |
| Aristocholiales | Aristolochiaceae | Ariatolochia | unidentified | radix aristolochiae | RT | 75% | 73% | 54% | 61% | 10% | *** | *** | *** |
| Asterales | Asteraccac | Artemisia | dracunculus | tarragon | | 77% | 100% | ** | 31% | −6% | 1.5 | 22 | 14.7 |
| Asterales | Asteraceae | Aster | unidentified | Radix asteris | RT | 79% | 94% | ** | 36% | −1% | 0.8 | 7.5 | 9.4 |
| Asterales | Asteraceac | Blumea | alata | | | 80% | 69% | 39% | 39% | 7% | *** | *** | *** |
| Asterales | Asteraceae | Cichorium | endivia | endive | | 81% | 100% | ** | 32% | 13% | 3.5 | 35 | 10 |
| Asterales | Asteraceae | Crassocephallum | mannii | | | 90% | 100% | ** | 35% | 24% | *** | *** | *** |
| Asterales | Asteraceae | Silybum | marianum | milk thistle | | 85% | 82% | 75% | 62% | 23% | *** | *** | *** |
| Asterales | Asteraceae | Sonchus | oleraceus | chicory | | 83% | 83% | ** | 28% | 4% | *** | *** | *** |
| Asterales | Asteracese | Taraxacum | mongolicum | mansen-tanpopo | PL | 75% | 100% | ** | 26% | 36% | *** | *** | *** |
| Asterales | Asteraceae | Taraxacum | officinale | dandelion | | 75% | 86% | ** | 19% | −2% | *** | *** | *** |
| Brassicales | Brassicaceae² | Brassica | rapa | turnip; choy sum | | 81% | 86% | ** | 29% | 27% | *** | *** | *** |
| Brassicales | Brassicaceae² | Capsella | bursa-pastoris | shepherd's purse | | 86% | 100% | ** | 30% | 38% | *** | *** | *** |
| Brassicales¹ | Brassicaceae² | Brassica | rapa | turnip | | 95% | 85% | 65% | 39% | 39% | *** | *** | *** |
| Cactales | Cactaceae | Hylocereus | undatus | pitahaya | FL | 76% | 91% | 65% | 45% | 43% | *** | *** | *** |
| Caryothyllales | Amaranthaceae | Alternanthera | pungens | burweed | | 86% | 81% | ** | 24% | 23% | *** | *** | *** |
| Caryophyllales | Caryophyllaceae | Stellaria | media | chickweed | | 80% | 98% | ** | 21% | 9% | 2 | 15 | 7.5 |
| Caryophyllales | Caryophyllaceae | Stellaria | media | chickweed | | 83% | 94% | 65% | 78% | 39% | 4 | 20 | 5 |
| Caryophyllales | Phytolaccaceae | Phytolacca | americana | pokeweed | | 80% | 76% | 58% | 5% | −8% | *** | *** | *** |
| Caryophyllales | Polygonacese | Polygonum | aviculare | michi-yanagi | | 76% | 81% | 47% | 28% | 26% | *** | *** | *** |
| Caryophyllales | Polygonaceae | Polygonum | caespitosum | hana-tade | | 78% | 85% | 46% | 33% | 30% | *** | *** | *** |
| Caryophyllales | Polygonaceae | Polygonum | odoratum | knotweed; smartweed | | 78% | 79% | 60% | 22% | 37% | *** | *** | *** |
| Caryophyllales | Polygonaceae | Polygonum | unidentified | PL | 75% | 76% | 43% | 55% | 1% | *** | *** | *** | |
| Caryophyllales | Polygonaceae | Rumex | japonicus | Japanese dock | | 81% | 100% | ** | 51% | 3% | 0.7 | 9 | 13.8 |
| Cucurbitales | Cucurbitaceae | Citrullus | vulgaris | watermelon | | 87% | 88% | 89% | 100% | 47% | *** | *** | *** |
| Cucurbitales | Cucurbitacese | Mukia | maderaspatana | cucumber | | 88% | 78% | ** | 30% | 26% | *** | *** | *** |
| Elaeagnales | Elaeagnaceae | Elaeagnus | umbellata | silver berry | | 82% | 86% | 81% | 56% | 50% | *** | *** | *** |
| Fagales | Fagaceae | Castanea | sativa | Spanish chestnut | SD | 79% | 85% | 83% | 50% | 51% | *** | *** | *** |
| Gnetales | Ginkgoaceae | Ginkgo | biloba | ginko nuts | | 83% | 100% | 79% | 53% | 50% | *** | *** | *** |
| Graminales | Poaceae³ | Coix | lacryma-jobi | Job's tears | | 76% | 81% | 60% | 29% | 7% | 2 | 35 | 17.5 |
| Graminales | Poaceae³ | Eleusine | coracana | sweet Indian millet | SD | 84% | 100% | ** | 48% | 47% | *** | *** | *** |
| Graminales | Poaceae³ | Hordeum | distichum | barley | | 80% | 100% | ** | 34% | 30% | *** | *** | *** |
| Graminales | Poaceae³ | Oryza | sativa | rice | | 78% | 74% | 54% | −20% | 12% | *** | *** | *** |
| Graminales | Poaceae³ | Oryza | sativa var. | sticky sweet rice | SD | 75% | 95% | ** | 16% | 20% | *** | *** | *** |
| | | | sticky sweet | | | | | | | | | | |
| Graminales | Poaceae³ | Zea | mays | corn | | 82% | 85% | 69% | 49% | 21% | *** | *** | *** |
| Lamiales | Lamiaceae⁴ | Lycopus | lucidus | herba lycopi | PL | 75% | 95% | ** | 15% | 1% | *** | *** | *** |
| Lamiales | Lamiaceae⁴ | Ocimum | basilicum | herba ocimi | PX | 75% | 74% | 40% | 33% | 21% | *** | *** | *** |
| Lamiales | Lamiaceae⁴ | Perilla | fructeacens | folium perillae | LF | 76% | 82% | 62% | 31% | 70% | *** | *** | *** |
| Lamiales | Lamiaceae⁴ | Prunella | vulgaris | spica prunellae | FL | 78% | 89% | ** | 26% | 42% | *** | *** | *** |
| Lamiales | Lamiaceae⁴ | Salvia | unidentified | sage | RT | 80% | 94% | ** | 44% | 62% | 3.5 | 18 | 5 |
| Lamiales | Verbenaceae | Vitex | agnus-castus | chaste lamb | | 82% | 78% | 47% | 40% | 0% | 1.5 | 50 | 33 |
| Liliales | Dioscoreaceac | Dioscorea | minutiflora | bush yam | | 79% | 95% | ** | 17% | 9% | 1.5 | 18 | 12 |
| Liliales | Dioscoreaceae | Dioscorea | unidentified | yam | RT | 77% | 59% | 36% | −7% | −4% | *** | *** | *** |
| Liliales | Liliaceae | Allium | schoenoprasum | Chinese chives | | 90% | 96% | 79% | 46% | 47% | *** | *** | *** |
| Liliales | Liliaceae | Allium | unidentified | | FR | 77% | 84% | ** | 20% | 3% | *** | *** | *** |
| Liliales | Liliaceae | Lilium | unidentified | lilly flower | FL | 81% | 91% | ** | 33% | 23% | *** | *** | *** |
| Liliales | Liliaceae | Smilax | omata | sarsaparilla | | 79% | 79% | ** | −24% | 3% | *** | *** | *** |
| Liliales | Liliaceae | Trillium | erectum | bethroot | | 79% | 72% | 43% | 55% | −5% | *** | *** | *** |
| Malvales | Malvaceae | Abutilon | unidentified | mallow seed | SD | 78% | 96% | ** | 24% | −13% | 1.5 | 28 | 18.7 |
| Malvales | Sterculiaceae | Sterculia | lychnophora | luoi uoi | FR | 81% | 82% | 58% | 31% | 45% | *** | *** | *** |
| Musales | Marantaceae | Maranta | arundinacea | arrowroot | | 79% | 100% | ** | 43% | −41% | 0.7 | 5 | 7.7 |
| Musales | Musaceae | Musa | paradisiaca | banana blossom | FL | 82% | 75% | 49% | 22% | 21% | *** | *** | *** |
| Myrtales | Balanphoraceae¹⁰ | Cynomorium | sangaricum | caulis cynomorii | ST | 83% | 99% | ** | 41% | −12% | 2 | 15 | 7.5 |
| Myrtales | Onagraceae | Oenothera | biennis | primrose | | 78% | 74% | 52% | 50% | 14% | *** | *** | *** |
| Papaverales | Capparidaceae | Capparis | spinosa | caper berries | FR | 80% | 86% | 66% | 33% | 32% | *** | *** | *** |
| Papaverales | Papaveraceae | Papaver | somniferum | poppy | | 80% | 95% | 90% | 81% | 65% | *** | *** | *** |
| Papaverales | Papaveraceae | Papaver | somniferum | poppy | SD | 87% | 100% | ** | 47% | −8% | 1.5 | 30 | 20 |
| Papaverales | Papaveraceae | Papaver | somniferum | poppy | FL | 79% | 80% | 60% | −42% | 24% | *** | *** | *** |
| Plantaginales | Plantaginaceae | Plantago | psyllium | psyllium | | 79% | 76% | ** | 19% | 18% | *** | *** | *** |
| Polemoniales⁷ | Boraginaceae⁵ | Cordia | tetrandra | | | 78% | 80% | ** | 18% | 23% | *** | *** | *** |
| Polemoniales⁷ | Convolvulaceae | Ipomoca | aquatica | water spinach | | 91% | 79% | ** | 38% | 29% | *** | *** | *** |
| Polemoniales⁷ | Solanaceae | Capsicum | annuum | pasilla Chile pepper | FR | 76% | 64% | 32% | 33% | 37% | *** | *** | *** |
| Polemoniales⁷ | Solanaceae | Capsicum | annuum | pepper | | 83% | 100% | ** | 69% | 64% | 0.75 | 8 | 10.7 |
| Polemoniales⁷ | Solanaceae | Capsicum | annuum | pepper | | 77% | 73% | 71% | 24% | 46% | *** | *** | *** |
| Polemoniales⁷ | Solanaceae | Capsicum | chinense | Chinese pepper | FR | 84% | 83% | 69% | 49% | 37% | *** | *** | *** |
| Polemoniales⁷ | Solanaceae | Solanum | melongena | eggplant | | 80% | 100% | ** | 65% | 68% | *** | *** | *** |
| Polemoniales⁷ | Solanaceae | Solanum | tuberosum | potato | | 78% | 100% | ** | 41% | 45% | 2 | 12 | 6 |
| Polemoniales⁷ | Solanaceae | Solanum | tuberosum | potato | | 76% | 100% | ** | 31% | 51% | *** | *** | *** |
| Polemoniales⁷ | Solanaceae | Solanum | tuberosum | potato | | 76% | 72% | ** | 16% | 5% | *** | *** | *** |
| Ranales | Menispermaceae | Cissampelos | mucronata | jenjoko; mugulita | | 75% | 99% | ** | 37% | 20% | 1.8 | 18 | 10 |
| Rosales | Fabaceae | Acacia | sieberiana | muwunga (Africa) | | 79% | 49% | ** | 27% | 9% | *** | *** | *** |
| Rosales | Fabaceae | Albizzia | julibrissin | mimosa | | 82% | 84% | 64% | 33% | 41% | *** | *** | *** |
| Rosales | Fabaceae | Glycine | max | soybean | SD | 76% | 89% | 85% | 53% | 55% | *** | *** | *** |
| Rosales | Fabaceae | Phaseolus | vulgaris var. | Peruvian bean | SD | 85% | 67% | 42% | 18% | 37% | *** | *** | *** |
| | | | Peruvian | | | | | | | | | | |
| Rosales | Fabaceae | Trigonella | foenum- | fenugreek | | 76% | 92% | ** | 25% | 34% | *** | *** | *** |
| | | | graecum | | | | | | | | | | |
| Rosales | Fabaceae | Vigna | umbellata | red bean | | 79% | 100% | ** | 32% | 25% | *** | *** | *** |
| Rosales | Fabaceae | Vigna | unguiculata | long bean | FR | 78% | 82% | 58% | 37% | 61% | *** | *** | *** |
| Rubiales | Rubiaceae | Asperula | odorata | woodruff | | 87% | 90% | 58% | 72% | 29% | 1.5 | 4 | 2.7 |
| Rubiales | Valerianaceae | Valeriana | officinalis | valerian root | RT | 82% | 100% | ** | 39% | 57% | *** | *** | *** |
| Rutales¹ | Rutaceaa | Citrus | limonia | lime | | 84% | 83% | ** | 29% | 7% | 1.5 | 35 | 23 |
| Rutales¹ | Rutaceae | Citrus | unidentified | | | 83% | 93% | ** | 21% | 12% | 0.7 | 15 | 21 |
| Scrophulariale | Acanthaceae | Acanthus | arboreus | otagalo | | 78% | 44% | ** | 23% | 11% | *** | *** | *** |
| Umbellales | Apiaceae⁵ | Angelica | sinensis | angelica; dong quai tea | | 76% | 89% | 87% | 100% | 52% | *** | *** | *** |
| Umbellales | Apiaceae⁵ | Carum | carvi | black caraway | | 92% | 81% | 83% | 47% | 53% | *** | *** | *** |
| Umbellales | Apiaceae⁵ | Centella | asiatica | gotu kola | | 75% | 69% | ** | 30% | −119% | *** | *** | *** |
| Umbellales | Apiaceae⁵ | Eryngium | foetidum | coyote culantro; | | 90% | 88% | 62% | 44% | 35% | *** | *** | *** |
| | | | fitweed | | | | | | | | | | |
| Umbellales | Apiaceae⁵ | Peucedanum | unidentified | | RT | 78% | 100% | ** | 33% | 12% | 0.9 | 4 | 4.4 |
| Urticales | Moraceae | Morus | alba | fructus mori; | FR | 80% | 88% | ** | 27% | 5% | 1 | 20 | 20 |
| | | | | gishi-gishi | | | | | | | | | |
| Urticales | Ulmaceae | Ulmus | rubra | slippery elm | | 75% | 60% | 31% | 18% | 28% | *** | *** | *** |
| Violales | Flacourtiaceae | Pangium | edule | kluwak; pakem | FR | 80% | 90% | 72% | 55% | 47% | *** | *** | *** |
| Violales | Passifloraceae | Passiflora | edulis | passion flower | PX | 86% | 65% | 45% | 2% | −10% | *** | *** | *** |

*Primary screen performed at three concentrations. Samples were not repeated in a COX-2 confirmation assay.
**No data due to assay error.
***Not tested.
¹Brassicales also classified as Sapindales or Rutales
²Brassicaceae also classified as Cruciferae
³Poaceae also classified as Graminae
⁴Lamiaceae also classified as Labiatae
⁵Apiaceae also classified as Umbelliferae
⁶Boraginaceae also classified as Cordiaceae or Ehretiaceae
⁷Polemoniales also classified as Solanales
⁹Pandanales also classified as Arales or Alismatales
¹⁰Balanplsoraceae also classified as Cynomoriaceae

The order, family, genus, and species of each plant whose extract was tested for COX-2 and COX-1 inhibitory activities are shown.

Table 2 below provides a description detailing the particular edible use of each plant extract tested for COX-2 inhibition as set-forth in Table 1. The plants are listed alphabetically according to genus. In addition, a comprehensive listing of references known to those generally skilled in the art is provided that details the edible consumption of these plants.

TABLE 2
Edible Uses of Plant Extracts

| | | | Isolate/ | Sample | | |
|---|---|---|---|---|---|---|
| Index | Scientific Name | Common Name | Chemical ID | ID | Extract # | Reference |
| 140 | Abutilon | Mallows. | 78916 | 914485 | | |
| | unidentified | Seeds | | | | |
| | | edible. | | | | |
| 96 | Acacia sieberiana | muwunga | 78486 | 914134 | | 2 |
| | | (African) | | | | |

Yields a clear gum of good quality. Used like gum Arabic as bulking agent.

| | | | | | |
|---|---|---|---|---|---|
| 1 | Acanthus arboreus | otagalo | 78487 | 914135 | 1, 2 |

Leaves are a masticatory.

| | | | | | |
|---|---|---|---|---|---|
| 17 | Acorus calamus | calamus root | 80328 | 922701 | 1, 2, 3, 4 |

Rootstock made into candy. Also used as flavoring for alcoholic drinks.

| | | | | | |
|---|---|---|---|---|---|
| 18 | Acorus gramineus | Shih-chang | 79050 | 914619 | 2 |

Rhizome is eaten.

| | | | | | |
|---|---|---|---|---|---|
| 129 | Albizzia julibrissin | mimosa | 76892 | 912334 | 2 |

Young leaves are eaten after being boiled down.

| | | | | | |
|---|---|---|---|---|---|
| 130 | Allium schoenoprasum | Chinese | 78569 | 914138 | 1, 2, 3, 4 |
| | | chives | | | |

Leaves eaten in salads, soups and omelets.

| | | | | | |
|---|---|---|---|---|---|
| 229 | Allium unidentified | Many species | 79513 | 914847 | |
| | | edible. | | | |
| 5 | Alternanthera | burweed | 78470 | 914119 | 1, 2 |
| | pungens | | | | |

Young leaves are eaten.

| | | | | | |
|---|---|---|---|---|---|
| 10 | Angelica sinensis | angelica, | 79771 | 922605 | 3 |
| | | Dong quai tea | | | |

Roots eaten in soups.

| | | | | | |
|---|---|---|---|---|---|
| 31 | Aristolochia | Leaves of | 79611 | 914945 | |
| | unidentified | contorta and | | | |
| | | debilis eaten | | | |
| | | boiled | | | |
| 32 | Aristolochia | Leaves of | 79611 | 915905 | |
| | unidentified | contorta and | | | |
| | | debilis eaten | | | |
| | | boiled | | | |
| 35 | Artemisia | tarragon | 78683 | 914252 | 3 |
| | dracunculus | | | | |

Leaves eaten baked or in salads

| | | | | | |
|---|---|---|---|---|---|
| 34 | Asclepias tuberosa | pleurisy root | 80399 | 922772 | 1, 2, 3 |

Pods when boiled are eaten; tender shoots are eaten as greens. Roots are consumed boiled

| | | | | | |
|---|---|---|---|---|---|
| 185 | Asperula odorata | woodruff | 80436 | 922809 | 1, 2, 3 |

This plant is used for flavoring a beverage.

| | | | | | |
|---|---|---|---|---|---|
| 37 | Aster unidentified | Young leaves | 78941 | 914510 | |
| | | of many | | | |
| | | species | | | |
| | | eaten. | | | |
| 75 | Blumea alata | Leaves of | 78477 | 914125 | |
| | | other species | | | |
| | | eaten. | | | |
| 63 | Brassica rapa | Choy sum | 78573 | 914142 | 1, 2, 3, 4 |
| 57 | Brassica rapa | turnip | 78567 | 914136 | 1, 2, 4 |

Roots are eaten fresh, grated, cooked, put in soup or pickled. Leaves are eaten.

| | | | | | |
|---|---|---|---|---|---|
| 65 | Capparis spinosa | Caper berries | 79419 | 914753 | 1, 2, 3, 4 |

Flower buds are eaten pickled.

| | | | | | |
|---|---|---|---|---|---|
| 58 | Capsella bursa- | shepherd's | 80400 | 922773 | 1, 2, 3 |
| | pastoris | purse | | | |

The plant is used as a vegetable.

| | | | | | |
|---|---|---|---|---|---|
| 206 | Capsicum annuum | pepper | 79789 | 922623 | 1, 2, 3, 4 |
| 207 | Capsicum annuum | pepper | 78583 | 914152 | 1, 2, 3, 4 |
| 212 | Capsicum annuum | Pasilla Chile | 78624 | 914193 | 1, 2, 3, 4 |
| | | pepper | | | |

Pods and young leaves are edible.

| | | | | | |
|---|---|---|---|---|---|
| 208 | Capsicum chinense | Chinese | 78581 | 914150 | 6 |
| | | pepper | | | |

The fruits are edible.

| | | | | | |
|---|---|---|---|---|---|
| 12 | Carum carvi | black caraway | 78630 | 914199 | 1, 2, 3, 4 |

Young shoots and leaves can be eaten. Seeds are used for flavoring.

| | | | | | |
|---|---|---|---|---|---|
| 254 | Castanea sativa | Spanish | 78865 | 914434 | 1, 2, 3, 4 |
| | | chestnut | | | |

fruits of most species edible

| | | | | | |
|---|---|---|---|---|---|
| 13 | Centella asiatica | gotu kola | 78454 | 914103 | 1, 2, 3, 4 |

The herb is eaten as a salad, also cooked in some countries.

| | | | | | |
|---|---|---|---|---|---|
| 38 | Cichorium endivia | endive | 78703 | 914272 | 3 |

Leaves for salad or as a boiled vegetable.

| | | | | | |
|---|---|---|---|---|---|
| 142 | Cissampelos | jenjoko, | 78485 | 914133 | 1, 3 |
| | mucronata | mugulita | | | |

Cited for food use in NAPRALLERT with no details.

| | | | | | |
|---|---|---|---|---|---|
| 77 | Citrullus vulgaris | watermelon | 79763 | 922597 | 2 |

Fruits are eaten ripe. Seeds are parched and eaten.

| | | | | | |
|---|---|---|---|---|---|
| 195 | Citrus limonia | lime | 78593 | 914162 | 3 |

The juice is used to add sour taste to foods. Also used in beverages.

| | | | | | |
|---|---|---|---|---|---|
| 196 | Citrus unidentified | Fruits of | 77669 | 912496 | |
| | | most are | | | |
| | | edible | | | |
| | | (oranges, | | | |
| | | limes, | | | |
| | | lemons, etc.) | | | |
| 163 | Coix lacryma-jobi | Job's tears | 80461 | 922834 | 1, 2, 3 |

Seeds are used as tea in Japan, Vietnam, etc. They are eaten as cereals in porridge, soups or pastries

| | | | | | |
|---|---|---|---|---|---|
| 22 | Colocasia esculenta | Malanga coco | 78076 | 912918 | 1, 2, 3, 4 |
| 23 | Colocasia esculenta | Dried taro | 79794 | 922628 | 1, 2, 3, 4 |
| | | stem | | | |

The tubers are eaten boiled, fried, steamed, put in soup (essential in New Year ceremonial
miso-soup in W. Honshu), pounded into dumplings or employed as a starch resource. Young
leaves and leaf stalks are eaten as vegetable or sun-dried for later use.

| | | | | | |
|---|---|---|---|---|---|
| 86 | Cordia tetrandra | Species not | 77182 | 912455 | |
| | | found, but | | | |
| | | the fruits of | | | |
| | | a number of | | | |
| | | species of | | | |
| | | this genus | | | |
| | | are edible. | | | |
| 39 | Crassocephallum | Possibly | 78469 | 914118 | |
| | mannii | Gynura | | | |
| | | mannii. | | | |
| | | Species not | | | |
| | | found, but | | | |
| | | leaves of | | | |
| | | other species | | | |
| | | edible. | | | |
| 82 | Cynomorium | Species not | 79013 | 914582 | |
| | sangaricum | found. Other | | | |
| | | species are | | | |
| | | condiments. | | | |
| 83 | Dioscorea | bush yam | 78483 | 914131 | 6 |
| | minutiflora | | | | |

Tubers (tubercules) are edible.

| | | | | | |
|---|---|---|---|---|---|
| 84 | Dioscorea | Yams. Most | 79323 | 914657 | |
| | unidentified | tubers edible | | | |
| 249 | Elaegnus umbellata | Silver | 76938 | 912365 | 5 |
| | | berries | | | |

Fruits edible scalded.

| | | | | | |
|---|---|---|---|---|---|
| 167 | Eleusine coracana | Sweet Indian | 79796 | 922630 | 1, 2, 3, 4 |
| | | millet | | | |

Cereal grain eaten.

| | | | | | |
|---|---|---|---|---|---|
| 14 | Eryngium foetidum | coyote | 78570 | 914139 | 1, 2, 3, 4 |
| | | culantro; | | | |
| | | fitweed | | | |

Roots as condiment in soups and meat dishes they impart a very agreeable flavor. Young
leaves are eaten raw, steamed or cooked with rice

| | | | | | |
|---|---|---|---|---|---|
| 117 | Ginkgo biloba | ginko nuts | 78610 | 914179 | 3 |

Seeds (nuts) are edible roasted or dried.

| | | | | | |
|---|---|---|---|---|---|
| 111 | Glycine max | Soy bean | 78995 | 914564 | 1, 2, 3, 4 |

Bean is eaten.

| | | | | | |
|---|---|---|---|---|---|
| 164 | Hordeum distichon | barley | 80506 | 922879 | 5 |

Cereal grain edible. Used in making beer.

| | | | | | |
|---|---|---|---|---|---|
| 64 | Hylocereus undatus | Pitahaya | 78839 | 914408 | 2 |

Fruit is edible.

| | | | | | |
|---|---|---|---|---|---|
| 76 | Ipomoea aquatica | water spinach | 78608 | 914177 | 1, 2, 3, 4 |

Leaves and young, tubular stems are used as vegetable.

| | | | | | |
|---|---|---|---|---|---|
| 134 | Lilium unidentified | Lilies. | 79331 | 914665 | |
| | | Bulbs of many | | | |
| | | species | | | |
| | | edible. | | | |
| 120 | Lycopus lucidus | Herba Lycopi | 79514 | 914848 | 2 |

Roots eaten boiled or in soup.

| | | | | | |
|---|---|---|---|---|---|
| 141 | Maranta arundinacea | arrowroot | 78867 | 914436 | 3 |

Tubers are eaten raw, roasted, grated into a coarse meal or made into arrowroot powder.

| | | | | | |
|---|---|---|---|---|---|
| 145 | Morus alba | Fructus Mori | 79019 | 914588 | 2, 4 |

Fruits are edible (mulberry).

| | | | | | |
|---|---|---|---|---|---|
| 78 | Mukia maderaspatana | Alternate | 78458 | 914107 | |
| | | name is | | | |
| | | Cucumis | | | |
| | | maderaspatana. | | | |
| | | These are | | | |
| | | cucumbers. | | | |
| | | most related | | | |
| | | species have | | | |
| | | edible fruit. | | | |
| 147 | Musa paradisiaca | Banana | 78578 | 914147 | 1, 2, 3 |
| | | blossom | | | |

Fruit and blossoms are edible.

| | | | | | |
|---|---|---|---|---|---|
| 121 | Ocimum basilicum | Herba Ocimi | 78971 | 914540 | 1, 3, 4 |

Basil is used as a flavoring.

| | | | | | |
|---|---|---|---|---|---|
| 152 | Oenothera biennis | primrose | 80412 | 922785 | 1, 2, 3 |
| | | flowers | | | |

Roots and the shoot are eaten; the latter is consumed as salad.

| | | | | | |
|---|---|---|---|---|---|
| 165 | Oryza sativa | rice, many | 79428 | 914762 | 1, 2, 3, 4 |
| | | varieties | | | |
| 166 | Oryza sativa var. | Sticky sweet | 79796 | 922630 | 1,2, 3, 4 |
| | sticky sweet | rice | | | |

It is boiled or steamed usually, though some nations often cook it with other vegetable or put
in soups. It is also made into cakes, pastries, puddings and starch, and also fermented
into intoxicating beverages, vinegar and miso.

| | | | | | |
|---|---|---|---|---|---|
| 228 | Pangium edule | Peeled kluwak | 79314 | 914648 | 2 |
| | | nut | | | |
| 154 | Papaver somniferum | poppy | 78646 | 914215 | 1, 2, 3, 4 |
| 155 | Papaver somniferum | poppy | 78612 | 914181 | 1, 2, 3, 4 |
| 156 | Papaver somniferum | poppy | 80445 | 922818 | |

Opium, one of the famous narcotics, is obtained from the milky juice of the capsule. In India,
beverages are prepared from it. Nursery plant is eaten as vegetable in China. Seeds are used
in sweetmeats, bakery food, confectionary, curries and the manufacture of an edible oil.

| | | | | | |
|---|---|---|---|---|---|
| 157 | Passiflora edulis | Passion | 79382 | 914716 | 1, 2, 3 |
| | | flower | | | |

Fruit is edible.

| | | | | | |
|---|---|---|---|---|---|
| 122 | Perilla fructescens | Folium | 78955 | 914524 | 5 |
| | | Perillae | | | |

Oil used in oriental cooking. Leaves are a flavoring.

| | | | | | |
|---|---|---|---|---|---|
| 227 | Peucedanum | Most species | 78939 | 914508 | |
| | unidentified | are | | | |
| | | medicinal, | | | |
| | | but leaves | | | |
| | | and tubers of | | | |
| | | some species | | | |
| | | edible. | | | |
| 107 | Phaseolus vulgaris | Peruvian bean | 79398 | 914732 | 1, 2, 3, 4 |
| | var. Peruvian | | | | |

Seeds and pods are edible.

| | | | | | |
|---|---|---|---|---|---|
| 158 | Phytolacca americana | Pokeweed | 80393 | 922766 | 1, 2, 3, 4 |

Young shoots are eaten as potherb. Fruit was used to color wine and confectionaries.

| | | | | | |
|---|---|---|---|---|---|
| 162 | Plantago psyllium | psyllium seed | 80408 | 922781 | 3 |

Sprouted seeds eaten in salads. Seeds yield nutritional oil. Seed husk mucilage used as thickener.

| | | | | | |
|---|---|---|---|---|---|
| 268 | Polygonum aviculare | Michi-yanagi | 76896 | 912336 | 2, 3, 4 |

Leaves edible.

| | | | | | |
|---|---|---|---|---|---|
| 269 | Polygonum | Hana-tade | 76928 | 912358 | 5 |
| | caespitosum | | | | |

Edible in soups

| | | | | | |
|---|---|---|---|---|---|
| 170 | Polygonum odoratum | knotweed | 78837 | 914406 | 1, 2, 3 |
| | | smartweed | | | |

A condiment for fish and meat.

| | | | | | |
|---|---|---|---|---|---|
| 232 | Polygonum | Many species | 79569 | 914903 | |
| | unidentified | edible. | | | |
| 123 | Prunella vulgaris | Spica | 79018 | 914587 | 1, 2, 3, 4 |
| | | prunellae | | | |

Cold-water infusion of the plant is a beverage

| | | | | | |
|---|---|---|---|---|---|
| 173 | Rumex japonicus | Japanese dock | 76821 | 912284 | 1, 2, 3 |

Leaves are eaten boiled, in soups or dried for later use. Seeds are mixed with rice or ground
into flour for making into dumplings.

| | | | | | |
|---|---|---|---|---|---|
| 124 | Salvia unidentified | Sages. Whole | 79492 | 914826 | |
| | | plant edible | | | |
| | | in most | | | |
| | | species. | | | |
| 43 | Silybum marianum | milk thistle | 79480 | 914814 | 1, 2, 3, 4 |
| | | tea | | | |

Young shoots are boiled and eaten in spring. Seeds are roasted into a coffee substitute.

| | | | | | |
|---|---|---|---|---|---|
| 136 | Smilax ornata | sarsaparilla | 80404 | 922777 | 6 |

Used as a flavoring for beverages.

| | | | | | |
|---|---|---|---|---|---|
| 214 | Solanum melongena | eggplant | 78835 | 914404 | 1, 2, 3, 4 |

Fruit is cooked, put in soup, eaten raw with rice, stewed, fried, or-roasted, baked or pickled.
One of the favorite culinary vegetables in the Far East.
Leaves are mixed with the rice bran and salt.

| | | | | | |
|---|---|---|---|---|---|
| 215 | Solanum tuberosum | potato | 79653 | 914987 | 1, 2, 3, 4 |
| 216 | Solanum tuberosum | potato | 79654 | 914988 | 1, 2, 3, 4 |
| 217 | Solanum tuberosum | potato | 79651 | 914985 | 1, 2, 3, 4 |

Tubers are eaten in salad when raw. They are eaten cooked, steamed, fried, mashed, otherwise
prepared into various dishes. They are an important staple food in many countries, also
the essential source of starch and alcohol.

| | | | | | |
|---|---|---|---|---|---|
| 44 | Sonchus oleraceus | chicory | 78466 | 914115 | 1, 2, 3, 4 |

Young leaves are eaten raw or parboiled and then cooked.

| | | | | | |
|---|---|---|---|---|---|
| 68 | Stellaria media | chickweed | 76809 | 912274 | 3 |
| 69 | Stellaria media | chickweed | 79762 | 922596 | 3 |

Young parts are used as boiled vegetable in time of scarcity.

| | | | | | |
|---|---|---|---|---|---|
| 230 | Sterculia | Luoi uoi | 78838 | 914407 | 2 |
| | lychnophora | | | | |

Seeds are made into a beverage.

| | | | | | |
|---|---|---|---|---|---|
| 47 | Taraxacum mongolicum | Mansen- | 79523 | 914857 | 2.4 |
| | | tanpopo | | | |

Leave eaten as boiled vegetable

| | | | | | |
|---|---|---|---|---|---|
| 46 | Taraxacum officinale | dandelion | 79478 | 914812 | 1, 2, 3, 4 |

Leaves are used in salads; sometimes bleached. Source of Dandelion wine. Roots
are eaten raw, boiled or in lieu of coffee.

| | | | | | |
|---|---|---|---|---|---|
| 110 | Trigonella foenum- | fenugreek | 78605 | 914174 | 1, 2, 3, 4 |
| | graecum | seed | | | |

Seeds are used to adulterate coffee, also for spice. Leaves and pods are used as vegetable.

| | | | | | |
|---|---|---|---|---|---|
| 137 | Trillium erectum | bethroot | 79018 | 914587 | 1, 2, 3, 4 |

Young leaves are eaten in salads and as a potherb.

| | | | | | |
|---|---|---|---|---|---|
| 225 | Ulmus rubra | slippery elm | 79479 | 914813 | 3 |
| | | bark tea | | | |

Powdered bark is edible. Sweet mucilaginous inner bard is chewed

| | | | | | |
|---|---|---|---|---|---|
| 236 | Valeriana | Valerian root | 79365 | 914699 | 1, 3, 4 |
| | officinalis | | | | |

Root used to flavor ice cream, etc. Also used as an herbal tea.

| | | | | | |
|---|---|---|---|---|---|
| 112 | Vigna umbellata | red bean | 78604 | 914173 | 1, 3 |

Young leaves and pods eaten steamed. Dried seed boiled and eaten with rice and soups.

| | | | | | |
|---|---|---|---|---|---|
| 113 | Vigna unguiculata | long bean | 78580 | 914149 | 1, 2, 3, 4 |

Seeds are edible steamed, boiled or stir-fried. Dried seeds used in soups.

| | | | | | |
|---|---|---|---|---|---|
| 273 | Vitex agnus-castus | Chaste lamb | 79481 | 914815 | 4 |

Fruits of most species edible.

| | | | | | |
|---|---|---|---|---|---|
| 26 | Xanthosoma | Malanga | 78574 | 914143 | 1, 2, 3, 4 |
| | sagittifolium | | | | |
| 27 | Xanthosoma | Malanga | 78575 | 914144 | 1, 2, 3, 4 |
| | sagittifolium | | | | |

Tubers are eaten like taro.

| | | | | | |
|---|---|---|---|---|---|
| 4 | Yucca elephantipes | Spanish | 77717 | 912504 | 3 |
| | | dagger; izote | | | |

Flowers and young stem tips are edible.

| | | | | | |
|---|---|---|---|---|---|
| 169 | Zea mays | corn | 79625 | 914959 | 1, 2, 3, 4 |

Major cereal crop.

### References

1. NAPRALERT (NATural Products ALERT), which currently contains the extracted information from over 116,000 scientific research articles and books from 1650 A.D. to the present. The NAPRALERT database is housed and maintained by the Program for Collaborative Research in the Pharmaceutical Sciences (PCRPS), within the Department of Medicinal Chemistry and Pharmacognosy, in the College of Pharmacy of the University of Illinois at Chicago, 833 South Wood Street (M/C 877), Chicago, Ill. 60612, U.S.A.

2. Tyozaburo Tanaka, (Edited by Sasuke Nakao) Tanaka's Cyclopedia of Edible Plants of the World, Keigaku Publishing Co., Tokyo, Japan, 1976.

This is a compendium of about 11,000 species of plants, including the essential wild species of the world. This book is considered to be one of the principle references on the world's edible plants.

3. Stephen Facciola, Cornucopia II: A Source Book of Edible Plants, Kampong Publications, Vista, Calif., 1998.

This book records the more than 3,000 species available in the U.S. and abroad.

4. James A. Duke, Database of Phytochemical Constituents of GRAS Herbs and Other Economic Plants, CRC Press, Boca Raton, Fla., 1992.

A database of approximately 1000 plants and 3000 compounds.

5. George Macdonald Hocking, Dictionary of Natural Products, , Plexus Publishing, Inc., Medford, N.J., 1997.

“Terms in the field of Pharmacognosy relating to natural medicinal and pharmaceutical materials and the plants, animals and minerals from which they are derived.” The work contains over 18,000 entries.

6. Enrique Sanchez-Monge, Flora Agricola: Taxonomia de las Magnoliofitas (Angiospermas) de interes agricola, con excepcion de las de aprovechamiento exclusivamente ornamental o forestall, Ministerio de Agriculture, Pesca y Alimentacion, Madrid, Spain, (date unknown).

An excellent reference work in Spanish with descriptions of plants, common names in many languages and commercial use of agricultural organisms of the world

7. Anthony R. Torkelson, The Cross Name Index to Medicinal Plants, Volumes!—IV, CRC Press, Boca Raton, Fla., (1998-1999).

8. Umberto Quattrocchi, CRC World Dictionary of Plant Names: Common Names, Scientific Names, Eponyms, Synonyms, and Etymology (Volumes 1-4), CRC Press, Boca Raton, Fla. (2000).

9. W³TROPICOS, a web site providing access to the Missouri Botanical Garden's VAST (VAScular Tropicos) nomenclatural database and associated authority files.

10. Webster's Ninth New Collegiate Dictionary, Merriam-Webster Inc., Springfield, Mass., (1983).

Tables 3-24 further illustrate the ability of certain extracts isolated from the families identified in Table 1 to selectively inhibit COX-2. A total of six different concentrations of the various extracts were tested for their ability to inhibit both COX-1 and COX-2. The IC₅₀ value for COX-1 and COX-2 was also determined and a selectivity ratio was then calculated as set forth above. FIGS. 1-22 are graphs that depict the data shown in Tables 3-24 as indicated.

TABLE 3
Extract isolated from Vitex agnus-castus

| | COX-1 | COX-2 |
|---|---|---|
| Amount of | Activity | Activity |
| Extract | Relative | Relative |
| (μg/ml) | to Control | to Control |
| 100 | 33% | Not |
| | | determined |
| 33.3 | 62% | 5% |
| 11.1 | Not | 13% |
| | determined | |
| 3.70 | 78% | 31% |
| 1.23 | 88% | 57% |
| 0.41 | 98% | 79% |
| IC₅₀ | IC₅₀ | COX-2 |
| (μg/ml) | (μg/ml) | Selectivity |
| COX-1 | COX-2 | Ratio |
| 50 | 1.5 | 33.3 |

TABLE 4
Extract isolated from Citrus limonia

| | COX-1 | COX-2 |
|---|---|---|
| Amount of | Activity | Activity |
| Extract | Relative | Relative |
| (μg/ml) | to Control | to Control |
| 100 | 19% | Not |
| | | determined |
| 33.3 | 52% | Not |
| | | determined |
| 11.1 | 70% | Not |
| | | determined |
| 3.70 | 79% | 22% |
| 1.23 | 92% | 51% |
| 0.41 | 98% | 69% |
| IC₅₀ | IC₅₀ | COX-2 |
| (μg/ml) | (μg/ml) | Selectivity |
| COX-1 | COX-2 | Ratio |
| 35 | 1.5 | 23.3 |

TABLE 5
Extract isolated from Citrus sp.

| | COX-1 | COX-2 |
|---|---|---|
| Amount of | Activity | Activity |
| Extract | Relative | Relative |
| (μg/ml) | to Control | to Control |
| 100 | 16% | 4% |
| 33.3 | 37% | 4% |
| 11.1 | Not | 7% |
| | determined | |
| 3.70 | 67% | 16% |
| 1.23 | 80% | 35% |
| 0.41 | 88% | 64% |
| IC₅₀ | IC₅₀ | COX-2 |
| (μg/ml) | (μg/ml) | Selectivity |
| COX-1 | COX-2 | Ratio |
| 15 | 0.7 | 21.4 |

TABLE 6
Extract isolated from Papaver somniferum

| | COX-1 | COX-2 |
|---|---|---|
| Amount of | Activity | Activity |
| Extract | Relative | Relative |
| (μg/ml) | to Control | to Control |
| 100 | 26% | Not |
| | | determined |
| 33.3 | 46% | Not |
| | | determined |
| 11.1 | 65% | 5% |
| 3.70 | 67% | 26% |
| 1.23 | 81% | 55% |
| 0.41 | 88% | 72% |
| IC₅₀ | IC₅₀ | COX-2 |
| (μg/ml) | (μg/ml) | Selectivity |
| COX-1 | COX-2 | Ratio |
| 30 | 1.5 | 20.0 |

TABLE 7
Extract isolated from Morus alba

| | COX-1 | COX-2 |
|---|---|---|
| Amount of | Activity | Activity |
| Extract | Relative | Relative |
| (μg/ml) | to Control | to Control |
| 100 | 33% | 5% |
| 33.3 | 45% | 9% |
| 11.1 | Not | 9% |
| | determined | |
| 3.70 | 68% | 20% |
| 1.23 | 80% | 44% |
| 0.41 | 103% | 71% |
| IC₅₀ | IC₅₀ | COX-2 |
| (μg/ml) | (μg/ml) | Selectivity |
| COX-1 | COX-2 | Ratio |
| 20 | 1 | 20.0 |

TABLE 8
Extract isolated from Abutilon sp.

| | COX-1 | COX-2 |
|---|---|---|
| Amount of | Activity | Activity |
| Extract | Relative | Relative |
| (μg/ml) | to Control | to Control |
| 100 | 23% | Not |
| | | determined |
| 33.3 | 44% | 5% |
| 11.1 | 74% | 7% |
| 3.70 | 76% | 35% |
| 1.23 | 89% | 54% |
| 0.41 | 113% | 82% |
| IC₅₀ | IC₅₀ | COX-2 |
| (μg/ml) | (μg/ml) | Selectivity |
| COX-1 | COX-2 | Ratio |
| 28 | 1.5 | 18.7 |

TABLE 9
Extract isolated from Coix lacryma

| | COX-1 | COX-2 |
|---|---|---|
| Amount of | Activity | Activity |
| Extract | Relative | Relative |
| (μg/ml) | to Control | to Control |
| 100 | 14% | Not |
| | | determined |
| 33.3 | 51% | 5% |
| 11.1 | Not | 11% |
| | determined | |
| 3.70 | 100% | 39% |
| 1.23 | 95% | 59% |
| 0.41 | 105% | 80% |
| IC₅₀ | IC₅₀ | COX-2 |
| (μg/ml) | (μg/ml) | Selectivity |
| COX-1 | COX-2 | Ratio |
| 35 | 2 | 17.5 |

TABLE 10
Extract isolated from Artemisia dracunculus

| | COX-1 | COX-2 |
|---|---|---|
| Amount of | Activity | Activity |
| Extract | Relative | Relative |
| (μg/ml) | to Control | to Control |
| 100 | 27% | Not |
| | | determined |
| 33.3 | 41% | 1% |
| 11.1 | 66% | 5% |
| 3.70 | 81% | 23% |
| 1.23 | 82% | 51% |
| 0.41 | 90% | 75% |
| IC₅₀ | IC₅₀ | COX-2 |
| (μg/ml) | (μg/ml) | Selectivity |
| COX-1 | COX-2 | Ratio |
| 22 | 1.5 | 14.7 |

TABLE 11
Extract isolated from Yucca elephantipes

| | COX-1 | COX-2 |
|---|---|---|
| Amount of | Activity | Activity |
| Extract | Relative | Relative |
| (μg/ml) | to Control | to Control |
| 100 | 4% | Not |
| | | determined |
| 33.3 | 28% | 3% |
| 11.1 | Not | 11% |
| | determined | |
| 3.70 | 66% | 32% |
| 1.23 | 79% | 56% |
| 0.41 | 105% | 80% |
| IC₅₀ | IC₅₀ | COX-2 |
| (μg/ml) | (μg/ml) | Selectivity |
| COX-1 | COX-2 | Ratio |
| 10 | 0.7 | 14.3 |

TABLE 12
Extract isolated from Rumex japonicus

| | COX-1 | COX-2 |
|---|---|---|
| Amount of | Activity | Activity |
| Extract | Relative to | Relative to |
| (ug/ml) | Control | Control |
| 100 | 10% | 1% |
| 33.3 | 30% | 3% |
| 11.1 | Not | 5% |
| | determined | |
| 3.70 | 63% | 15% |
| 1.23 | 72% | 35% |
| 0.41 | 88% | 62% |
| IC₅₀ | IC₅₀ | COX-2 |
| (ug/ml) | (ug/ml) | Selectivity |
| COX-1 | COX-2 | Ratio |
| 9 | 0.65 | 13.8 |

TABLE 13
Extract isolated from Dioscorea minutiflora

| | COX-1 | COX-2 |
|---|---|---|
| Amount of | Activity | Activity |
| Extract | Relative to | Relative to |
| (ug/ml) | Control | Control |
| 100 | Not | Not |
| | determined | determined |
| 33.3 | 18% | Not |
| | | determined |
| 11.1 | 69% | Not |
| | | determined |
| 3.70 | 90% | 24% |
| 1.23 | 95% | 50% |
| 0.41 | 109% | 70% |
| IC₅₀ | IC₅₀ | COX-2 |
| (ug/ml) | (ug/ml) | Selectivity |
| COX-1 | COX-2 | Ratio |
| 18 | 1.5 | 12.0 |

TABLE 14
Extract isolated from Capsicum annuum

| | COX-1 | COX-2 |
|---|---|---|
| Amount of | Activity | Activity |
| Extract | Relative to | Relative to |
| (ug/ml) | Control | Control |
| 100 | 16% | 7% |
| 33.3 | 26% | 9% |
| 11.1 | 41% | 11% |
| 3.70 | 72% | 18% |
| 1.23 | 99% | 38% |
| 0.41 | 112% | 65% |
| IC₅₀ | IC₅₀ | COX-2 |
| (ug/ml) | (ug/ml) | Selectivity |
| COX-1 | COX-2 | Ratio |
| 8 | 0.75 | 10.7 |

TABLE 15
Extract isolated from Cissampelos mucronata

| | COX-1 | COX-2 |
|---|---|---|
| Amount of | Activity | Activity |
| Extract | Relative to | Relative to |
| (ug/ml) | Control | Control |
| 100 | 9% | Not |
| | | determined |
| 33.3 | 35% | Not |
| | | determined |
| 11.1 | 58% | 8% |
| 3.70 | 72% | 34% |
| 1.23 | 83% | 58% |
| 0.41 | 98% | 83% |
| IC₅₀ | IC₅₀ | COX-2 |
| (ug/ml) | (ug/ml) | Selectivity |
| COX-1 | COX-2 | Ratio |
| 18 | 1.8 | 10.0 |

TABLE 16
Extract isolated from Cichorium endivia

| | COX-1 | COX-2 |
|---|---|---|
| Amount of | Activity | Activity |
| Extract | Relative to | Relative to |
| (ug/ml) | Control | Control |
| 100 | 9% | 2% |
| 33.3 | 51% | 8% |
| 11.1 | Not | 27% |
| | determined | |
| 3.70 | 93% | 46% |
| 1.23 | 98% | 78% |
| 0.41 | 104% | 98% |
| IC₅₀ | IC₅₀ | COX-2 |
| (ug/ml) | (ug/ml) | Selectivity |
| COX-1 | COX-2 | Ratio |
| 35 | 3.5 | 10.0 |

TABLE 17
Extract isolated from Aster sp.

| | COX-1 | COX-2 |
|---|---|---|
| Amount of | Activity | Activity |
| Extract | Relative to | Relative to |
| (ug/ml) | Control | Control |
| 100 | Not | Not |
| | determined | determined |
| 33.3 | 17% | Not |
| | | determined |
| 11.1 | Not | 1% |
| | determined | |
| 3.70 | 66% | 23% |
| 1.23 | 78% | 40% |
| 0.41 | 90% | 69% |
| IC₅₀ | IC₅₀ | COX-2 |
| (ug/ml) | (ug/ml) | Selectivity |
| COX-1 | COX-2 | Ratio |
| 7.5 | 0.8 | 9.4 |

TABLE 18
Extract isolated from Maranta arundinacea

| | COX-1 | COX-2 |
|---|---|---|
| Amount of | Activity | Activity |
| Extract | Relative to | Relative to |
| (ug/ml) | Control | Control |
| 100 | Not | Not |
| | determined | determined |
| 33.3 | 7% | Not |
| | | determined |
| 11.1 | 26% | Not |
| | | determined |
| 3.70 | 57% | 10% |
| 1.23 | 65% | 34% |
| 0.41 | 82% | 60% |
| IC₅₀ | IC₅₀ | COX-2 |
| (ug/ml) | (ug/ml) | Selectivity |
| COX-1 | COX-2 | Ratio |
| 5 | 0.65 | 7.7 |

TABLE 19
Extract isolated from Cynomorium sangaricum

| | COX-1 | COX-2 |
|---|---|---|
| Amount of | Activity | Activity |
| Extract | Relative to | Relative to |
| (ug/ml) | Control | Control |
| 100 | 7% | Not |
| | | determined |
| 33.3 | 31% | Not |
| | | determined |
| 11.1 | 57% | 3% |
| 3.70 | 75% | 37% |
| 1.23 | 74% | 57% |
| 0.41 | 84% | 75% |
| IC₅₀ | IC₅₀ | COX-2 |
| (ug/ml) | (ug/ml) | Selectivity |
| COX-1 | COX-2 | Ratio |
| 15 | 2 | 7.5 |

TABLE 20
Extract isolated from Solanum tuberosum

| | COX-1 | COX-2 |
|---|---|---|
| Amount of | Activity | Activity |
| Extract | Relative to | Relative to |
| (ug/ml) | Control | Control |
| 100 | 13% | 7% |
| 33.3 | 27% | 14% |
| 11.1 | 50% | 19% |
| 3.70 | 82% | 31% |
| 1.23 | 96% | 62% |
| 0.41 | 102% | 86% |
| IC₅₀ | IC₅₀ | COX-2 |
| (ug/ml) | (ug/ml) | Selectivity |
| COX-1 | COX-2 | Ratio |
| 12 | 2 | 6.0 |

TABLE 21
Extract isolated from Salvia sp.

| | COX-1 | COX-2 |
|---|---|---|
| Amount of | Activity | Activity |
| Extract | Relative to | Relative to |
| (ug/ml) | Control | Control |
| 100 | 15% | 8% |
| 33.3 | 27% | 10% |
| 11.1 | 64% | 22% |
| 3.70 | 85% | 47% |
| 1.23 | 95% | 80% |
| 0.41 | 107% | 88% |
| IC₅₀ | IC₅₀ | COX-2 |
| (ug/ml) | (ug/ml) | Selectivity |
| COX-1 | COX-2 | Ratio |
| 18 | 3.5 | 5.1 |

TABLE 22
Extract isolated from Stellaria media

| | COX-1 | COX-2 |
|---|---|---|
| Amount of | Activity | Activity |
| Extract | Relative | Relative to |
| (ug/ml) | to Control | Control |
| 100 | 13% | 8% |
| 33.3 | 27% | 12% |
| 11.1 | 71% | 23% |
| 3.70 | 82% | 51% |
| 1.23 | 99% | 86% |
| 0.41 | 126% | 115% |
| IC₅₀ | IC₅₀ | COX-2 |
| (ug/ml) | (ug/ml) | Selectivity |
| COX-1 | COX-2 | Ratio |
| 20 | 4 | 5.0 |

TABLE 23
Extract isolated from Peucedanum sp.

| | COX-1 | COX-2 |
|---|---|---|
| Amount of | Activity | Activity |
| Extract | Relative | Relative to |
| (ug/ml) | to Control | Control |
| 100 | 3% | 1% |
| 33.3 | 23% | 5% |
| 11.1 | Not | 12% |
| | determined | |
| 3.70 | 51% | 25% |
| 1.23 | 70% | 41% |
| 0.41 | 88% | 69% |
| IC₅₀ | IC₅₀ | Cox-2 |
| (ug/ml) | (ug/ml) | Selectivity |
| COX-1 | COX-2 | Ratio |
| 4 | 0.9 | 4.4 |

TABLE 24
Extract isolated from Asperula odorata

| | COX-1 | COX-2 |
|---|---|---|
| Amount of | Activity | Activity |
| Extract | Relative | Relative to |
| (ug/ml) | to Control | Control |
| 100 | Not | Not |
| | determined | determined |
| 33.3 | 1% | 5% |
| 11.1 | 28% | 6% |
| 3.70 | 52% | 26% |
| 1.23 | 68% | 55% |
| 0.41 | 74% | 84% |
| IC₅₀ | IC₅₀ | COX-2 |
| (ug/ml) | (ug/ml) | Selectivity |
| COX-1 | COX-2 | Ratio |
| 4 | 1.5 | 2.7 |

As illustrated by these data, the organic extracts isolated from the indicated edible plant inhibit COX-2. In fact, several of the extracts selectively inhibit COX-2 over COX-1 by greater than 10-fold. In view of the above, it will be seen that the several objectives of the invention are achieved and other advantageous results attained.

What is claimed is:

1. A method for selective inhibition of COX-2 in an organism, the method comprising the step of administering to the organism a composition comprising a therapeutically or prophylactically effective amount of an organic extract of an edible plant, wherein the inhibitory effect of the extract on COX-2 activity is greater than or equal to about 2 times greater than the inhibitory effect of the extract on COX-1 activity.

2. The method of claim 1 wherein the COX-2 activity is greater than or equal to about 10 times greater than the inhibitory effect of the extract on COX-1 activity.

3. A method for inhibiting the activity of COX-2 in an organism, the method comprising the step of administering to the organism a composition comprising a therapeutically or prophylactically effective amount of an organic extract of an edible plant, wherein the plant is selected from the orders consisting of Agavales, Apocynales, Arales, Aristolochiales, Asterales, Brassicales, Cactales, Caryophyllales, Cucurbitales, Elaeagnales, Fagales, Gnetales, Graminales, Lamiales, Liliales, Malvales, Musales, Myrtales, Papaverales, Plantaginales, Polemoniales, Ranales, Rosales, Rubiales, Rutales, Scrophulariales, Umbellales, Urticales, and Violales.

4. The method of claim 3 wherein the inhibitory effect of the extract on COX-2 activity is greater than or equal to about 2 times greater than the inhibitory effect of the extract on COX-1 activity.

5. The method of claim 3 wherein the inhibitory effect of the extract on COX-2 activity is greater than or equal to about 10 times greater than the inhibitory effect of the extract on COX-1 activity.

6. The method of claim 3 wherein the organic extract of the Agavales order is selected from the plant family Agavaceae.

7. The method of claim 6 wherein the organic extract of the Agavaceae family is from the genus Yucca.

8. The method of claim 3 wherein the organic extract of the Apocynales order is selected from the plant family Asclepiadaceae.

9. The method of claim 8 wherein the organic extract of the Asclepiadaceae family is from the genus Asclepias.

10. The method of claim 3 wherein the organic extract of the Arales order is selected from the plant family Araceae.

11. The method of claim 10 wherein the organic extract of the Araceae family is selected from the genera consisting of Acorus, Colocasia, and Xanthosoma.

12. The method of claim 3 wherein the organic extract of the Aristolochiales order is selected from the plant family Aristolochiaceae.

13. The method of claim 12 wherein the organic extract of the Aristolochiaceae family is from the genus Aristolochia.

14. The method of claim 3 wherein the organic extract of the Asterales order is selected from the plant family Asteraceae.

15. The method of claim 14 wherein the organic extract of the Asteraceae family is selected from the genera consisting of Artemisia, Aster, Blumea, Cichorium, Crassocephallum, Silybum, Sonchus, and Taraxacum.

16. The method of claim 3 wherein the organic extract of the Brassicales order is selected from the plant family Brassicaceae.

17. The method of claim 16 wherein the organic extract of the Brassicaceae family is selected from the genera consisting of Brassica and Capsella.

18. The method of claim 3 wherein the organic extract of the Cactales order is selected from the plant family Cactaceae.

19. The method of claim 18 wherein the organic extract of the Cactaceae family is from the genus Hylocereus.

20. The method of claim 3 wherein the organic extract of the Caryophyllales order is selected from the plant families consisting of Amaranthaceae, Caryophyllaceae, Phytolaccaceae and Polygonaceae.

21. The method of claim 20 wherein the organic extract of the Amaranthaceae family is from the genus Alternanthera.

22. The method of claim 20 wherein the organic extract of the Caryophyllaceae family is from the genus Stellaria.

23. The method of claim 20 wherein the organic extract of the Phytolaccaceae family is from the genus Phytolacca.

24. The method of claim 20 wherein the organic extract of the Polygonaceae family is selected from the genera consisting of Polygonum and Rumex.

25. The method of claim 3 wherein the organic extract of the Cucurbitales order is selected from the plant family Cucurbitaceae.

26. The method of claim 25 wherein the organic extract of the Cucurbitaceae family is selected from the genera consisting of Citrullus and Mukia.

27. The method of claim 3 wherein the organic extract of the Eleagnales order is selected from the plant family Elaeagnaceae.

28. The method of claim 27 wherein the organic extract of the Elaeagnaceae family is selected from the genus consisting of Elaeagnus.

29. The method of claim 3 wherein the organic extract of the Fagales order is selected from the plant family Fagaceae.

30. The method of claim 29 wherein the organic extract of the Fagaceae family is selected from the genus consisting of Castanea.

31. The method of claim 3 wherein the organic extract of the Gnetales order is selected from the plant family Ginkgoaceae.

32. The method of claim 31 wherein the organic extract of the Ginkgoaceae family is from the genus Ginkgo.

33. The method of claim 3 wherein the organic extract of the Graminales order is selected from the plant family Poaceae.

34. The method of claim 33 wherein the organic extract of the Poaceae family is selected from the genera consisting of Coix, Eleusine, Hordeum, Oryza, and Zea.

35. The method of claim 3 wherein the organic extract of the Lamiales order is selected from the plant families Lamiaceae and Verbenaceae.

36. The method of claim 35 wherein the organic extract of the Lamiaceae family is selected from the genera consisting of Lycopus, Ocimum, Perilla, Prunella and Salvia.

37. The method of claim 35 wherein the organic extract of the Verbenaceae family is selected from the genus consisting of Vitex.

38. The method of claim 3 wherein the organic extract of the Liliales order is selected from the plant families consisting of Dioscoreaceae and Liliaceae.

39. The method of claim 38 wherein the organic extract of the Dioscoreaceae family is from the genus Dioscorea.

40. The method of claim 38 wherein the organic extract of the Liliaceae family is selected from the genera consisting of Allium, Lilium, Smilax, and Trillium.

41. The method of claim 3 wherein the organic extract of the Malvales order is selected from the plant family Malvaceae and Sterculiaceae.

42. The method of claim 41 wherein the organic extract of the Malvaceae family is from the genus Abutilon.

43. The method of claim 41 wherein the organic extract of the Sterculiaceae family is from the genus Sterculia.

44. The method of claim 3 wherein the organic extract of the Musales order is selected from the plant families consisting of Marantaceae and Musaceae.

45. The method of claim 44 wherein the organic extract of the Marantaceae family is from the genus Maranta.

46. The method of claim 44 wherein the organic extract of the Musaceae family is from the genus Musa.

47. The method of claim 3 wherein the organic extract of the Myrtales order is selected from the plant families consisting of Balanphoraceae and Onagraceae.

48. The method of claim 47 wherein the organic extract of the Balanphoraceae family is from the genus Cynomorium.

49. The method of claim 47 wherein the organic extract of the Onagraceae family is from the genus Oenothera.

50. The method of claim 3 wherein the organic extract of the Papaverales order is selected from the plant families consisting of Capparidaceae and Papaveraceae.

51. The method of claim 50 wherein the organic extract of the Capparidaceae family is from the genus Capparis.

52. The method of claim 50 wherein the organic extract of the Papaveraceae family is from the genus Papaver.

53. The method of claim 3 wherein the organic extract of the Plantaginales order is selected from the plant family Plantaginaceae.

54. The method of claim 53 wherein the organic extract of the Plantaginaceae family is from the genus Plantago.

55. The method of claim 3 wherein the organic extract of the Polemoniales order is selected from the plant families consisting of Boraginaceae, Convolvulaceae, and Solanaceae.

56. The method of claim 55 wherein the organic extract of the Boraginaceae family is from the genus Cordia.

57. The method of claim 55 wherein the organic extract of the Convolvulaceae family is from the genus Ipomoea.

58. The method of claim 55 wherein the organic extract of the Solanaceae family is selected from the genera consisting of Capsicum and Solanum.

59. The method of claim 3 wherein the organic extract of the Ranales order is selected from the plant family Menispermaceae.

60. The method of claim 59 wherein the organic extract of the Menispermaceae family is from the genus Cissampelos.

61. The method of claim 3 wherein the organic extract of the Rosales order is selected from the plant family Fabaceae.

62. The method of claim 61 wherein the organic extract of the Fabaceae family is selected from the genera consisting of Acacia, Albizzia, Glycine, Phaseolus, Trigonella, and Vigna.

63. The method of claim 3 wherein the organic extract of the Rubiales order is selected from the plant families consisting of Rubiaceae and Valerianaceae.

64. The method of claim 63 wherein the organic extract of the Rubiaceae family is from the genus Asperula.

65. The method of claim 63 wherein the organic extract of the Valerianaceae family is from the genus Valeriana.

66. The method of claim 3 wherein the organic extract of the Rutales order is selected from the plant family Rutaceae.

67. The method of claim 66 wherein the organic extract of the Rutaceae family is from the genus Citrus.

68. The method of claim 3 wherein the organic extract of the Scrophulariales order is selected from the plant family Acanthaceae.

69. The method of claim 68 wherein the organic extract of the Acanthaceae family is from the genus Acanthus.

70. The method of claim 3 wherein the organic extract of the Umbellales order is selected from the plant family Apiaceae.

71. The method of claim 70 wherein the organic extract of the Apiaceae family is selected from the genera consisting of Angelica, Carum, Centella, Eryngium, and Peucedanum.

72. The method of claim 3 wherein the organic extract of the Urticales order is selected from the plant families consisting of Moraceae and Ulmaceae.

73. The method of claim 72 wherein the organic extract of the Moraceae family is from the genus Morus.

74. The method of claim 72 wherein the organic extract of the Ulmaceae family is from the genus Ulmus.

75. The method of claim 3 wherein the organic extract of the Vioales order is selected from the plant families Flacourtiaceae and Passifloraceae.

76. The method of claim 75 wherein the organic extract of the Flacourtiaceae family is from the genus Pangium.

77. The method of claim 75 wherein the organic extract of the Passifloraceae family is from the genus Passiflora.

78. The method of claim 1 wherein the organic extract is a purified composition obtained by a method comprising:
(a) contacting the plant with an organic solvent to remove an extract from the plant wherein the extract inhibits COX-2 activity; and
(b) isolating the extract with COX-2 inhibitory activity.

79. The method of claim 78 wherein the extract selectively inhibits COX-2 activity.

80. The method of claim 78 wherein step (a) further comprises mixing the plant with the organic solvent and stirring the resulting mixture at a temperature between about 25°0 C. and the boiling point of said solvent for at least one minute.

81. The method of claim 78 wherein the organic solvent is selected from the group consisting of hydrocarbon solvents, ethers, chlorinated solvents, acetone, ethyl acetate, butanol, ethanol, methanol, isopropyl alcohol and mixtures thereof.

82. The method of claim 81 wherein the organic solvent is non-polar.

83. The method of claim 82 wherein the non-polar organic solvent is dichloromethane or hexane.

84. The method of claim 78 wherein step (b) further comprises separating the solvent from the organic extract by evaporating the solvent.

85. A method of treating or preventing COX-2 mediated inflammation or an inflammation-associated disorder in an organism, the method comprising administering to the organism a composition comprising a therapeutically or prophylactically effective amount of the purified composition according to claim 78.

86. The method of claim 85 wherein the inflammation-associated disorder is arthritis.

87. The method of claim 85 wherein the inflammation-associated disorder is pain.

88. The method of claim 85 wherein the inflammation-associated disorder is fever.

89. The method of claim 85 for use in the treatment or prevention of cancer.

90. The method of claim 89 wherein the cancer is epithelial cell cancer.

91. The method of claim 90 wherein the epithelial cell cancer is colon, breast, prostate, bladder, or lung cancer.

92. The method of claim 85 for use in the treatment or prevention of central nervous system disorders.

93. The method of claim 92 wherein the central nervous system disorder is Alzheimer's Disease.
